(19) United States
(12) Patent Application Publication
(10) Pub. No.: US 2008/0113975 A1
(43) Pub. Date: May 15, 2008

(54) THIOUREA COMPOUNDS

(75) Inventors: Jyh-Haur Chern, Taipei (TW); Tsu-An Hsu, Taipei (TW); Iou-Jiun Kang, Wandan Township (TW); Li-Wen Wang, Kaohsiung City (TW); Chung-Chi Lee, Chung-He City (TW); Yen-Chun Lee, Taitung City (TW); Yu-Sheng Chao, Warren, NJ (US)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 11/839,326

(22) Filed: Aug. 15, 2007

Related U.S. Application Data
(60) Provisional application No. 60/837,782, filed on Aug. 15, 2006.

(51) Int. Cl. A61K 31/145; A61K 31/24; A61K 31/341; A61K 31/381; A61K 31/40; A61K 31/404; A61K 31/44; A61K 31/451; A61K 31/47; A61K 31/495; A61K 31/5375; A61K 31/54; A61P 31/12; C07C 335/18; C07C 335/20; C07D 207/09; C07D 207/327; C07D 209/04; C07D 213/63; C07D 215/20; C07D 215/233; C07D 217/02; C07D 241/04; C07D 279/12; C07D 295/14; C07D 295/155; C07D 307/54; C07D 333/28

(52) U.S. Cl. 514/227.5; 514/237.8; 514/255.03; 514/307; 514/311; 514/312; 514/331; 514/351; 514/415; 514/427; 514/428; 514/438; 514/471; 514/539; 514/586; 514/587; 544/160; 544/395; 544/58.1; 546/146; 546/153; 546/175; 546/231; 546/300; 548/504; 548/561; 548/567; 549/496; 549/77; 560/16; 564/27; 564/28

(57) ABSTRACT

This invention relates to thiourea compounds of formula (I) shown below: 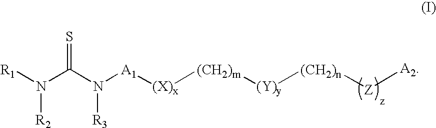 Each variable in formula (I) is defined in the specification. These compounds can be used to treat hepatitis C virus infection.

### CROSS REFERENCE

Pursuant to 35 U.S.C. § 119(e), this application claims priority to U.S. Provisional Application 60/837,782, filed on Aug. 15, 2006. The contents of the provisional application are incorporated by reference.

### BACKGROUND

Hepatitis C virus (HCV) infection is estimated to affect 170 million individuals worldwide. This disease is primarily transmitted through contaminated blood products. Although its spread has been slowed as a result of improvement in blood screening in many countries, it remains the leading cause of liver disease-related deaths in the world. For example, it causes about 10,000 deaths annually in the U.S. alone. In the absence of effective therapies, the death rate is expected to triple over the next 2 decades.

Current treatments based on interferon-alpha have low success rates, particularly for genotype-1 infections predominant in Europe, Japan, and the U.S. Also, they are expensive and poorly received by patients. Thus, there is a need to develop better therapeutic agents for treating HCV infection.

### SUMMARY

This invention is based on the discovery that certain thiourea compounds are effective in treating hepatitis C virus infection.

In one aspect, this invention relates to thiourea compounds of formula (I): 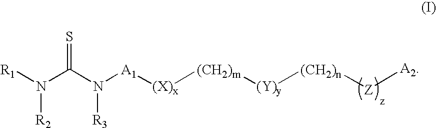
In this formula, each of R₁, R₂, and R₃, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; or R₁ and R₂, together with the nitrogen atom to which they are bonded, are C₃-C₂₀ heterocycloalkyl; or R₂ and R₃, together with the two nitrogen atoms to which they are bonded and the carbon atom bonded to both of the two nitrogen atoms, are C₃-C₂₀ heterocycloalkyl; each of A₁ and A₂, independently, is aryl or heteroaryl; each of X, Y, and Z, independently, is O, S, S(O), S(O)₂, N(Rₐ), C(RₐR_{b}), C₁-C₁₀ alkyl, C₂-C₁₀ alkenyl, C₂-C₁₀ alkynyl, C₃-C₂₀ cycloalkyl, C₁-C₂₀ heterocycloalkyl, aryl, or heteroaryl, in which each of Rₐ and R_{b}, independently, is H, C₁-C₁₀ alkyl, C₃-C₂₀cycloalkyl, C₁-C₂₀ heterocycloalkyl, aryl, or heteroaryl; each of m and n, independently, is 1, 2, 3, 4, or 5; and each of x, y, and z, independently, is 0 or 1.

Referring to formula (I), a subset of the thiourea compounds described above are those in which x is 1, y is 0, and z is 0. In these compounds, X can be O or NH, A₁ can be phenylene, A₂ can be phenyl, and each of R₁, R₂, and R₃, independently, can be H or C₁-C₁₀ alkyl optionally substituted with aryl.

Another subset of the thiourea compounds described above are those in which x is 1, y is 0, and z is 1. In these compounds, X and Z can both be O, each of R₁, R₂, and R₃ can be H, or R₁ and R₂, together with the nitrogen atom to which they are bonded, can be C₃-C₂₀ heterocycloalkyl, A₁ can be phenylene, and A₂ can be heteroaryl, or aryl optionally substituted with halo, aryl, heteroaryl, CN, OR, COOR, or NRR′, in which each of R and R′ independently, is H, C₁-C₁₀ alkyl, or aryl. Referring to formula (I), another subset of the thiourea compounds described above are those in which x is 1, y is 1, and z is 1. In these compounds, X and Z can both be O, Y can be C(RₐR_{b}) (in which each of Rₐ and R_{b}, independently, can be C₁-C₁₀ alkyl), A₁ can be phenylene, A₂ can be phenyl optionally substituted with aryl, and each of R₁, R₂, and R₃ can be H.

The term “alkyl” refers to a saturated, linear or branched, hydrocarbon moiety, such as —CH₃, —CH(CH₃)₂, or —CH₂—. The term “alkenyl” refers to a linear or branched hydrocarbon moiety that contains at least one double bond, such as —CH═CH—CH₃ or —CH═CH—CH₂—. The term “alkynyl” refers to a linear or branched hydrocarbon moiety that contains at least one triple bond, such as —C≡C—CH₃ or —C≡C—CH₂—. The term “cycloalkyl” refers to a saturated, cyclic hydrocarbon moiety, such as cyclohexyl or cyclohexylene. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl or 4-tetrahydropyranylene. The term “heterocycloalkenyl” refers to a non-aromatic, cyclic moiety having at least one ring heteroatom (e.g., N, O, or S) and at least one double bond, such as pyranyl. The term “aryl” refers to a hydrocarbon, moiety having one or more aromatic rings. Examples of aryl moieties include phenyl (Ph), phenylene, naphthyl, naphthylene, pyrenyl, anthryl, and phenanthryl 1. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀, alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

In another aspect, this invention features thiourea compounds of formula (I), in which R₁ is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; each of R₂ and R₃, independently, is C₁-C₁₀ alkyl, C₂-C₁₀ alkenyl, C₂-C₁₀ alkynyl, C₃-C₂₀cycloalkyl, C₃-C₂₀ cycloalkenyl, C₁-C₂₀ heterocycloalkyl, C₁-C₂₀ heterocycloalkenyl, aryl, or heteroaryl; or R₂ and R₃, together with the two nitrogen atoms to which they are bonded and the carbon atom bonded to both of the two nitrogen atoms, are C₃-C₂₀heterocycloalkyl; each of A₁ and A₂, independently, is aryl or heteroaryl; each of X, Y, and Z, independently, is O, S, S(O), S(O)₂, N(Rₐ), C(RₐR_{b}), C₁-C₁₀ alkyl, C₂-C₁₀ alkenyl, C₂-C₁₀ alkynyl, C₃-C₂₀ cycloalkyl, C₁-C₁₀ heterocycloalkyl, aryl, or heteroaryl, in which each of Rₐ and R_{b}, independently, is H, C₁-C₁₀ alkyl, C₃-C₂₀ cycloalkyl, C₁-C₂₀ heterocycloalkyl, aryl, or heteroaryl; each of m and n, independently, is 0, 1, 2, 3, 4, or 5; and each of x, y, and z, independently, is 0 or 1.

Referring to formula (I), a subset of the thiourea compounds described above are those in which x is 1, y is 0, and z is 0. In these compounds, X can be O, A₁ can be phenylene, A₂ can be phenyl, R₁ can be H or C₁-C₁₀ alkyl optionally substituted with aryl, and R₂ and R₃, together with the two nitrogen atoms to which they are bonded and the carbon atom bonded to both of the two nitrogen atoms, can be C₃-C₂₀ heterocycloalkyl;

In another aspect, this invention relates to thiourea compounds of formula (II): 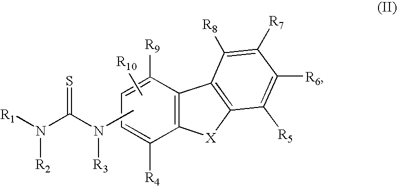
wherein X is O, N(Rₐ), C(RₐR_{b}), or C(O); each of R₁, R₂, and R₃, independently, is H, C₁-C₁₀ alkyl C₂-C₁₀ alkenyl, C₂-C₁₀ alkynyl, C₃-C₂₀ cycloalkyl, C₃-C₂₀ cycloalkenyl C₁-C₂₀ heterocycloalkyl, C₁-C₂₀ heterocycloalkenyl, aryl, or heteroaryl; or R₂ and R₃, together with the two nitrogen atoms to which they are bonded and the carbon atom bonded to both of the two nitrogen atoms, are C₃-C₂₀ heterocycloalkyl; and each of R₄, R₅, R₆, R₇, R₈, R₉, and R₁₀ independently, is H, C₁-C₁₀ alkyl C₂-C₁₀ alkenyl, C₂-C₁₀ alkynyl, C₃-C₂₀cycloalkyl C₃-C₂₀ cycloalkenyl, C₁-C₂₀ heterocycloalkyl, C₁-C₂₀ heterocycloalkenyl aryl, heteroaryl halo, N(R_{c}R_{d), N(R}_{c})—C(S)—N(R_{d}Rₑ); N(R_{c})—C(O)R_{d}, or N(Rₑ)—C(O)O—R_{d}; in which each of Rₐ, R_{b}, R_{c}, R_{d}, and Rₑ, independently, is H, C₁-C₁₀ alkyl C₃-C₂₀cycloalkyl, C₁-C₂₀ heterocycloalkyl, aryl, or heteroaryl; provided that if R₁₀ is at the 3-position, then 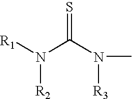
is at the 4-position; and if R₁₀ is at the 4-position, then 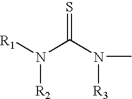
is at the 3-position. The 3- and 4-positions of the above formula are delineated below:

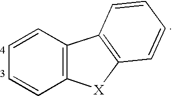

An embodiment of the just-described compounds features the following formula: 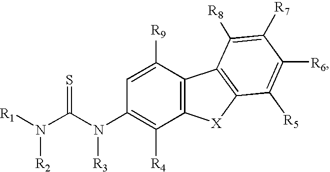
wherein X, R₁, R₂, R₃, R₄, R₅, R₆, R₇, R₈, and R₉ are as just defined.

Referring the above formula, a subset of the thiourea compounds described above are those in which each of R₁, R₂, and R₃, independently, is H, aryl optionally substituted with C₁-C₂₀ heterocycloalkyl, heteroaryl, or C₁-C₁₀ alkyl optionally substituted with C₁-C₁₀ alkoxy, aryl, N(RR′), in which each of R and R′, independently, is H or C₁-C₁₀ alkyl. In these compounds, each of R₄, R₅, R₆, R₇, R₈, and R₉, independently, can be H, halo, N(R_{c}R_{d}), N(R_{c})—C(S)—N(R_{d}Rₑ); N(R_{c})—C(O)R_{d}, or N(R_{c})—C(O)O—R_{d}. For example, each of R₄, R₅, R₇, R₈, and R₉ can be H and R₆, can be H, halo, N(R_{c}R_{d}), N(R_{c})—C(S)—N(R_{d}Rₑ), N(R_{c})—C(O)R_{d}, or N(R_{c})—C(O)O—R_{d}.

Another subset of the thiourea compounds described above are those in which each of R₁, R₂, and R₃ is H; or R₁ is (CH₂)ₙCH₃, in which n is 1, 2, 3, 4, 5, or 6, and each of R₂ and R₃ is H.

In still another aspect, this invention features a method for treating hepatitis C virus infection. The method includes administering to a subject in need thereof an effective amount of one or more thiourea compounds of formula (I) or (II) shown above. The term “treating” or “treatment” refers to administering one or more thiourea compounds to a subject, who has an above-described infection, a symptom of such an infection, or a predisposition, toward such an infection, with the purpose to confer a therapeutic effect, e.g., to cure, relieve, alter, affect, ameliorate, or prevent the above-described infection, the symptom of it, or the predisposition toward it.

In addition, this invention encompasses a pharmaceutical composition that contains an effective amount of at least one of the above-mentioned thiourea compounds and a pharmaceutically acceptable carrier.

The thiourea compounds described above include the compounds themselves, as well as their salts, prodrugs, and solvates, if applicable. A salt, for example, can be formed between an anion and a positively charged group (e.g., amino) on a thiourea compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a thiourea compound. Suitable cations include sodium ion, potassium ion, magnesium ion, calcium ion, and an ammonium cation such as tetramethylammonium ion. The thiourea compounds also include those salts containing quaternary nitrogen atoms. Examples of prodrugs include esters and other pharmaceutically acceptable derivatives, which, upon administration to a subject, are capable of providing active thiourea compounds. A solvate refers to a complex formed between, an active thiourea compound and a pharmaceutically acceptable solvent. Examples of pharmaceutically acceptable solvents include water, ethanol, isopropanol, ethyl acetate, acetic acid, and ethanolamine.

Also within the scope of this invention is a pharmaceutical composition containing one or more of the above-described thiourea compounds for use in treating HCV infection, as well as this therapeutic use and use of the compounds for the manufacture of a medicament for treating HCV infection.

The details of one or more embodiments of the invention are set forth in the description below. Other features, objects, and advantages of the invention will be apparent from the description and from the claims.

### DETAILED DESCRIPTION

The table below show 183 exemplary compounds of this invention:

TABLE 1

| | | | Molec- |
|---|---|---|---|
| Com- | | | ular |
| pound | | | Weight |
| No. | Structure | Name | (M + 1) |
| 1 | 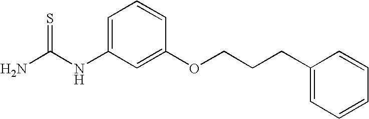 | [3-(3-Phenyl-propoxy)- phenyl]-thiourea | 287 |
| 2 | 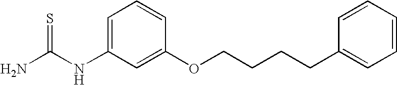 | [3-(4-Phenyl-butoxy)- phenyl]-thiuorea | 301 |
| 3 | 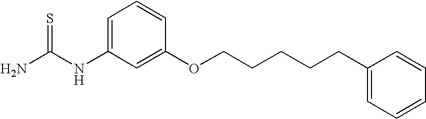 | [3-(5-Phenyl- pentyloxy)-phenyl]- thiourea | 315 |
| 4 | 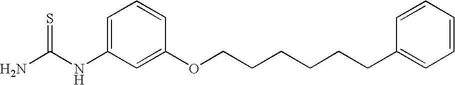 | [3-(6-Phenyl- hexyloxy)-phenyl]- thiourea | 329 |
| 5 | 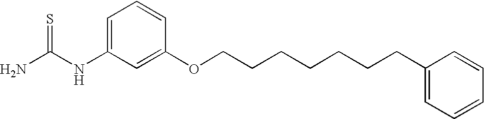 | [3-(7-Phenyl- heptyloxy)-phenyl]- thiourea | 343 |
| 6 | 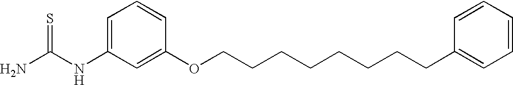 | [3-(8-Phenyl- octyloxy)-phenyl]- thiourea | 357 |
| 7 | 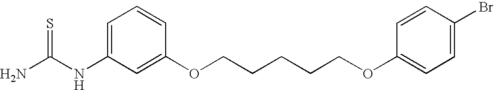 | {3-[5-(4-Bromo- phenoxy)-pentyloxy]- phenyl}-thiourea | 409 411 |
| 8 | 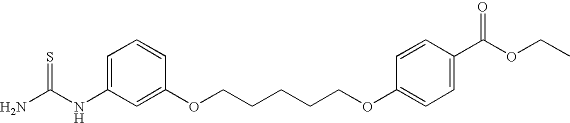 | 4-[5-(3-Thioureido- phenoxy)-pentyloxy]- benzoic acid ethyl ester | 403 |
| 9 | 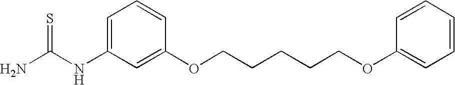 | [3-(5-Phenoxy- pentyloxy)-phenyl]- thiourea | 331 |
| 10 | 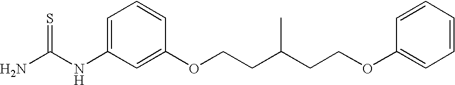 | [3-(3-Methyl-5- phenoxy-pentyloxy)- phenyl]-thiourea | 345 |
| 11 | 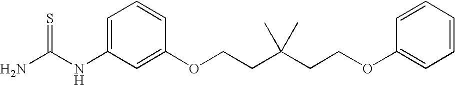 | [3-(3,3-Dimethyl-5- phenoxy-pentyloxy)- phenyl]-thiourea | 359 |
| 12 | 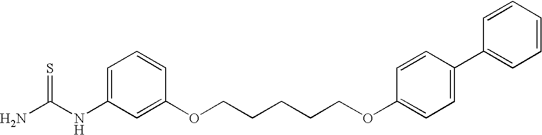 | {3-[5-(Biphenyl-4- yloxy)-pentyloxy]- phenyl}-thiourea | 407 |
| 13 | 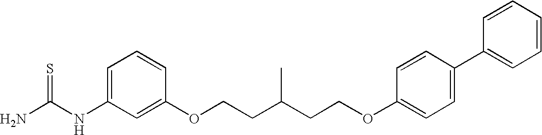 | {3-[5-(Biphenyl-4- yloxy)-3-methyl- pentyloxy]-phenyl}- thiourea | 421 |
| 14 | 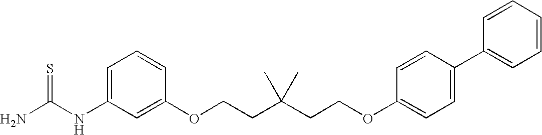 | {3-[5-(Biphenyl-4- yloxy)-3,3-dimethyl- pentyloxy]-phenyl}- thiourea | 435 |
| 15 | 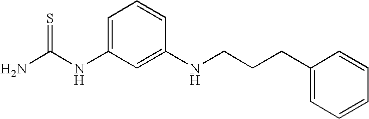 | [3-(3-Phenyl- propylamino)-phenyl]- thiourea | 286 |
| 16 | 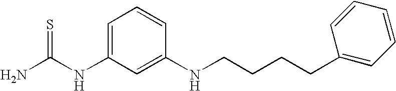 | [3-(4-Phenyl- butylamino)-phenyl]- thiourea | 300 |
| 17 | 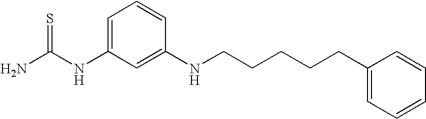 | [3-(5-Phenyl- pentylamino)-phenyl]- thiourea | 314 |
| 18 | 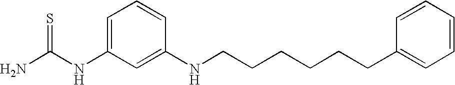 | [3-(6-Phenyl- hexylamino)-phenyl]- thiourea | 328 |
| 19 | 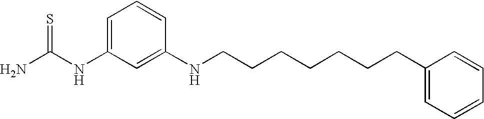 | [3-(7-Phenyl- heptylamino)-phenyl]- thiourea | 342 |
| 20 | 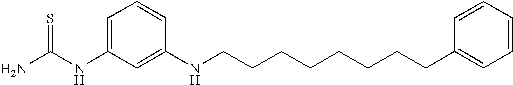 | [3-(8-Phenyl- octylamino)-phenyl]- thiourea | 356 |
| 21 | 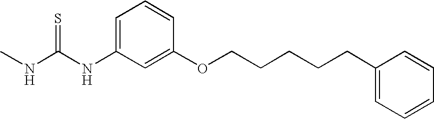 | 1-Methyl-3-[3-(5- phenyl-pentyloxy)- phenyl]-thiourea | 329 |
| 22 | 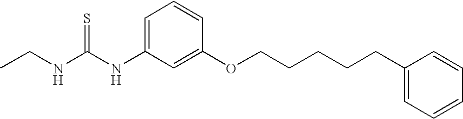 | 1-Ethyl-3-[3-(5- phenyl-pentyloxy)- phenyl]-thiourea | 343 |
| 23 | 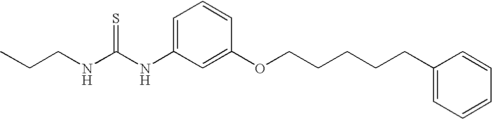 | 1-[3-(5-Phenyl- pentyloxy)-phenyl]-3- propyl-thiourea | 357 |
| 24 | 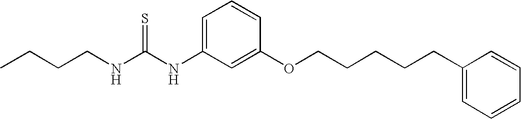 | 1-Butyl-3-[3-(5- phenyl-pentyloxy)- phenyl]-thiourea | 371 |
| 25 | 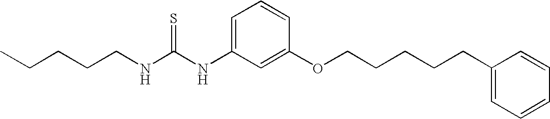 | 1-Pentyl-3-[3-(5- phenyl-pentyloxy)- phenyl]-thiourea | 385 |
| 26 | 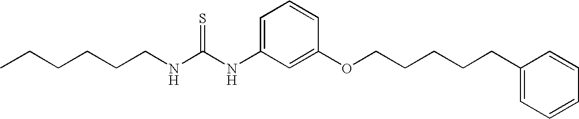 | 1-Hexyl-3-[3-(5- phenyl-pentyloxy)- phenyl]-thiourea | 399 |
| 27 | 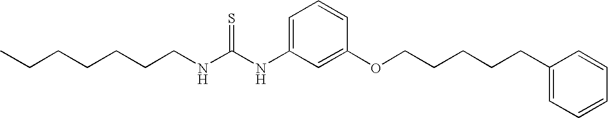 | 1-Heptyl-3-[3-(5- phenyl-pentyloxy)- phenyl]-thiourea | 413 |
| 28 | 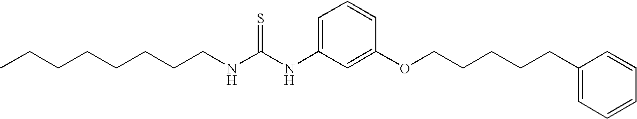 | 1-Octyl-3-[3-(5- phenyl-pentyloxy)- phenyl]-thiourea | 427 |
| 29 | 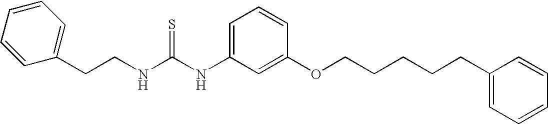 | 1-Phenethyl-3-[3-(5- phenyl-pentyloxy)- phenyl]-thiourea | 419 |
| 30 | 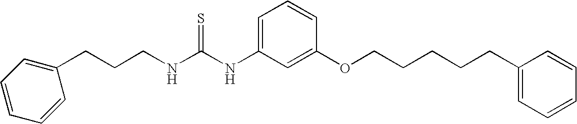 | 1-[3-(5-Phenyl- pentyloxy)-phenyl]-3- (3-phenyl-propyl)- thiourea | 433 |
| 31 | 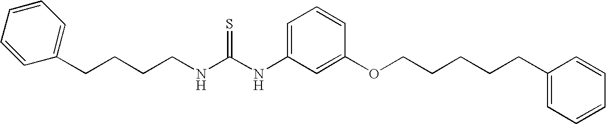 | 1-(4-Phenyl-butyl)-3- [3-(5-phenyl- pentyloxy)-phenyl]- thiourea | 447 |
| 32 | 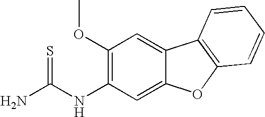 | (2-Methoxy- dibenzofuran-3-yl)- thiourea | 273 |
| 33 | 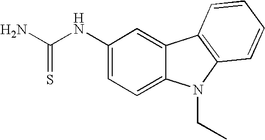 | (9-Ethyl-9H-carbazol- 3-yl)-thiourea | 270 |
| 34 | 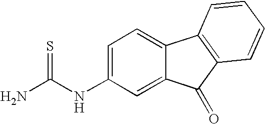 | (9-Oxo-9H-fluoren-2- yl)-thiourea | 255 |
| 35 | 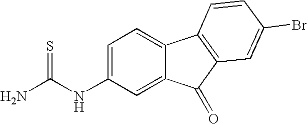 | (7-Bromo-9-oxo-9H- fluoren-2-yl)-thiourea | 332 334 |
| 36 | 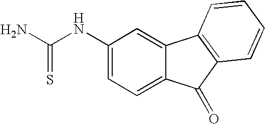 | (9-Oxo-9H-fluoren-3- yl)-thiourea | 255 |
| 37 | 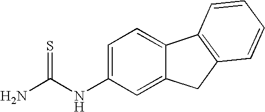 | (9H-Fluoren-2-yl)- thiourea | 241 |
| 38 | 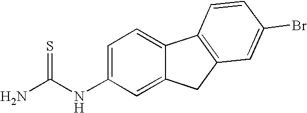 | (7-Bromo-9H-fluoren- 2-yl)-thiourea | 320 |
| 39 | 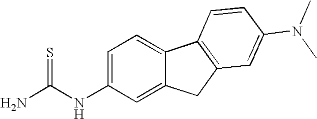 | (7-Dimethylamino-9H- fluoren-2-yl)-thiourea | 284 |
| 40 | 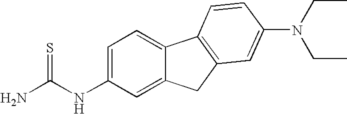 | (7-Diethylamino-9H- fluoren-2-yl)-thiourea | 312 |
| 41 | 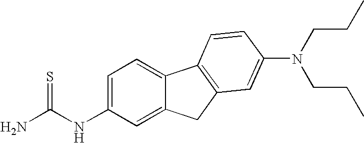 | (7-Dipropylamino-9H- fluoren-2-yl)-thiourea | 340 |
| 42 | 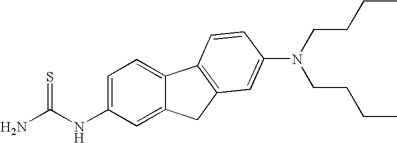 | (7-Dibutylamino-9H- fluoren-2-yl)-thiourea | 368 |
| 43 | 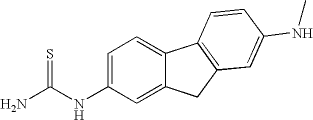 | (7-Methylamino-9H- fluoren-2-yl)-thiourea | 270 |
| 44 | 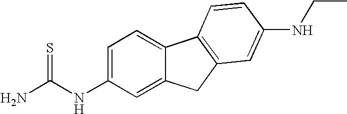 | (7-Ethylamino-9H- fluoren-2-yl)-thiourea | 284 |
| 45 | 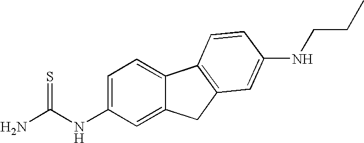 | (7-Propylamino-9H- fluoren-2-yl)-thiourea | 298 |
| 46 | 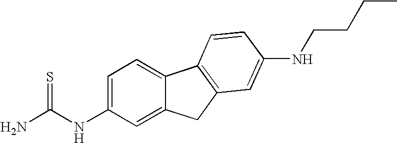 | (7-Butylamino-9H- fluoren-2-yl)-thiourea | 312 |
| 47 | 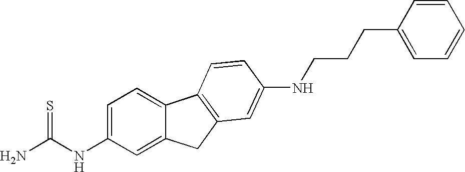 | [7-(3-Phenyl- propylamino)-9H- fluoren-2-yl]-thiourea | 374 |
| 48 | 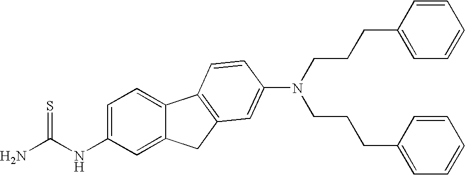 | {7-[Bis(3-phenyl- propyl)-amino]-9H- fluoren-2-yl}-thiourea | 492 |
| 49 | 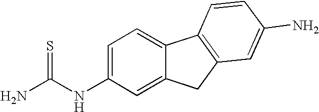 | (7-Amino-9H-fluoren- 2-yl)-thiourea | 256 |
| 50 | 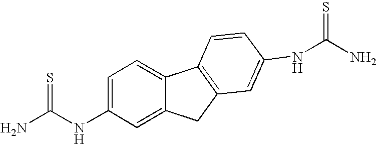 | (7-Thioureido-9H- fluoren-2-yl)-thiourea | 315 |
| 51 | 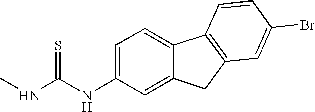 | 1-(7-Bromo-9H- fluoren-2-yl)-3-methyl- thiourea | 333 335 |
| 52 | 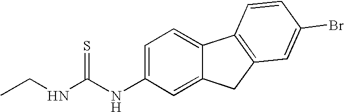 | 1-(7-Bromo-9H- fluoren-2-yl)-3-ethyl- thiourea | 347 349 |
| 53 | 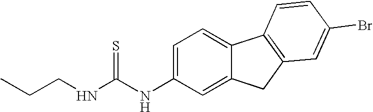 | 1-(7-Bromo-9H- fluoren-2-yl)-3-propyl- thiuorea | 361 363 |
| 54 | 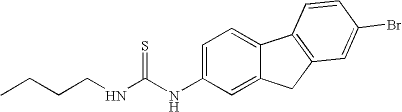 | 1-(7-Bromo-9H- fluoren-2-yl)-3-butyl- thiuorea | 375 377 |
| 55 | 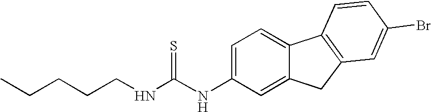 | 1-(7-Bromo-9H- fluoren-2-yl)-3-pentyl- thiuorea | 389 391 |
| 56 | 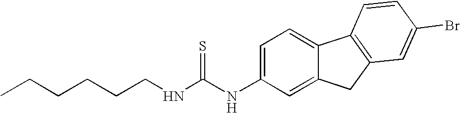 | 1-(7-Bromo-9H- fluoren-2-yl)-3-hexyl- thiuorea | 403 405 |
| 57 | 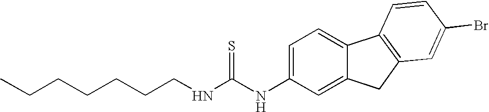 | 1-(7-Bromo-9H- fluoren-2-yl)-3-heptyl- thiuorea | 417 419 |
| 58 | 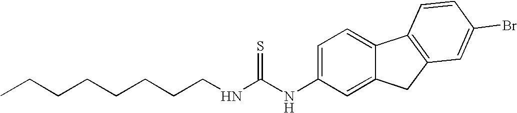 | 1-(7-Bromo-9H- fluoren-2-yl)-3-octyl- thiuorea | 431 433 |
| 59 | 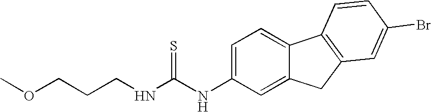 | 1-(7-Bromo-9H- fluoren-2-yl)-3-(3- methoxy-propyl)- thiourea | 391 393 |
| 60 | 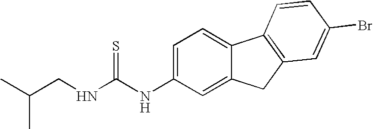 | 1-(7-Bromo-9H- fluoren-2-yl)-3- isobutyl-thiourea | 375 377 |
| 61 | 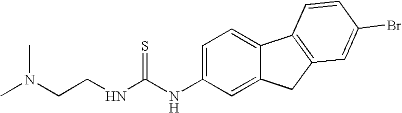 | 1-(7-Bromo-9H- fluoren-2-yl)-3-(2- dimethylamino-ethyl)- thiuorea | 390 392 |
| 62 | 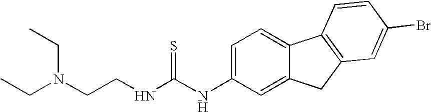 | 1-(7-Bromo-9H- fluoren-2-yl)-3-(2- diethylamino-ethyl)- thiuorea | 418 420 |
| 63 | 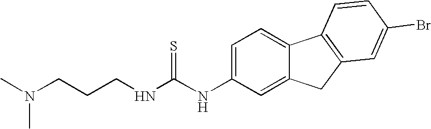 | 1-(7-Bromo-9H- fluoren-2-yl)-3-(2- dimethylamino- propyl)-thiourea | 404 406 |
| 64 | 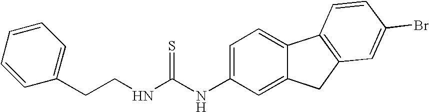 | 1-(7-Bromo-9H- fluoren-2-yl)-3-(3- phenethyl-thiourea | 423 425 |
| 65 | 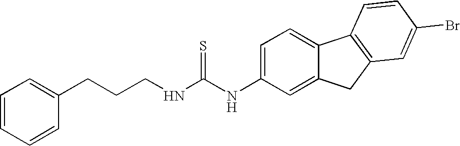 | 1-(7-Bromo-9H- fluoren-2-yl)-3-(3- phenyl-propyl)- thiuorea | 437 439 |
| 66 | 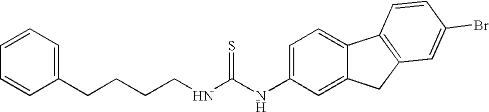 | 1-(7-Bromo-9H- fluoren-2-yl)-3-(4- phenyl-butyl)-thiourea | 451 453 |
| 67 | 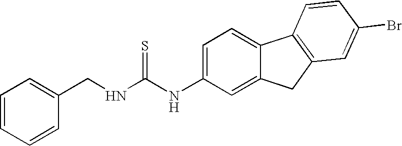 | 1-Benzyl-3-(7-bromo- 9H-fluoren-2-yl)- thiourea | 430 432 |
| 68 | 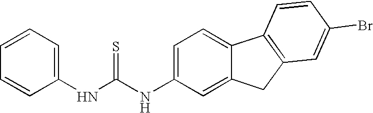 | 1-(7-Bromo-9H- fluoren-2-yl)-3-phenyl- thiuorea | 394 396 |
| 69 | 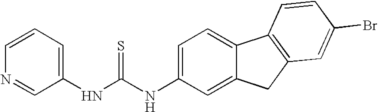 | 1-(7-Bromo-9H- fluoren-2-yl)-3- pyridin-3-yl-thiourea | 395 397 |
| 70 | 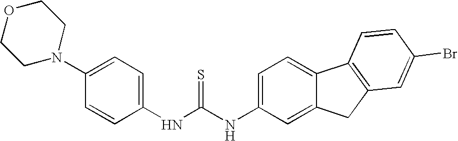 | 1-(7-Bromo-9H- fluoren-2-yl)-3-(4- morpholin-4-yl- phenyl)-thiourea | 480 482 |
| 71 | 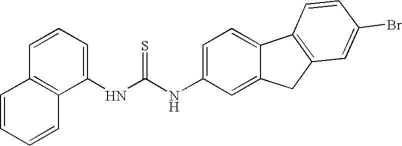 | 1-(7-Bromo-9H- fluoren-2-yl)-3- naphthalen-1-yl- thiourea | 445 447 |
| 72 | 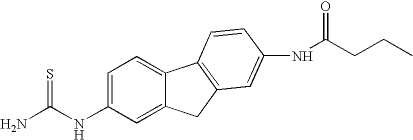 | N-(7-Thioureido-9H- fluoren-2-yl)- butyramide | 326 |
| 73 | 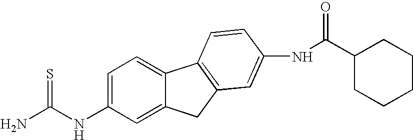 | Cyclohexanecarboxylic acid (7-thioureido-9H- fluoren-2-yl)-amide | 366 |
| 74 | 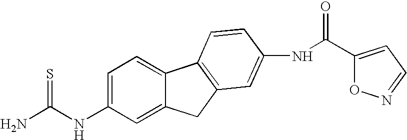 | Isoxazole-5-carboxylic acid (7-thioureido-9H- fluoren-2-yl)-amide | 351 |
| 75 | 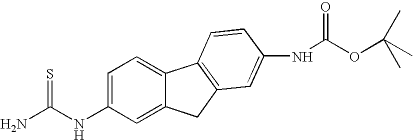 | (7-Thioureido-9H- fluoren-2-yl)-carbamic acid tert-butyl ester | 356 |
| 76 | 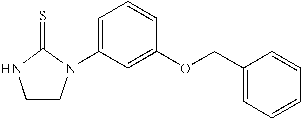 | 1-(3-Benzyloxy- phenyl)-imidazolidine- 2-thione | 285 |
| 77 | 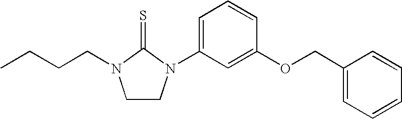 | 1-(3-Benzyloxy- phenyl)-3-butyl- imidazolidine-2-thione | 341 |
| 78 | 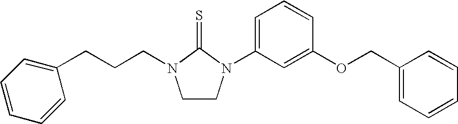 | 1-(3-Benzyloxy- phenyl)-3-(3-phenyl- propyl)-imidazolidine- 2-thione | 403 |
| 79 | 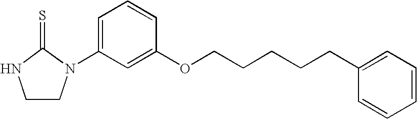 | 1-[3-(5-Phenyl- pentyloxy)-phenyl]- imidazolidine-2-thione | 341 |
| 80 | 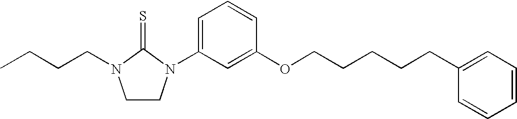 | 1-Butyl-3-[3-(5- phenyl-pentyloxy)- phenyl]-imidazolidine- 2-thione | 397 |
| 81 | 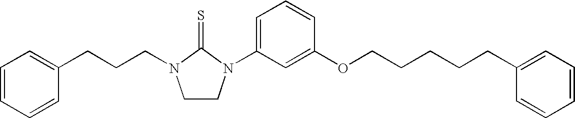 | 1-[3-(5-Phenyl- pentyloxy)-phenyl]-3- (3-phenyl-propyl)- imidazolidine-2-thione | 459 |
| 82 | 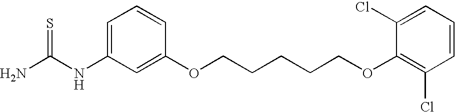 | {3-[5-(2,6-Dichloro- phenoxy)-pentyloxy]- phenyl}-thiourea | 400 |
| 83 | 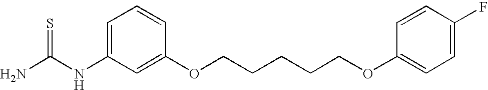 | {3-[5-(4-Fluoro- phenoxy)-pentyloxy]- phenyl}-thiourea | 349 |
| 84 | 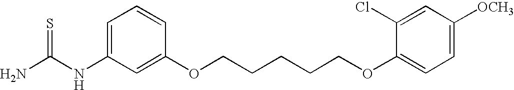 | {3-[5-(2-Chloro-4- methoxy-phenoxy)- pentyloxy]-phenyl}- thiourea | 395 |
| 85 | 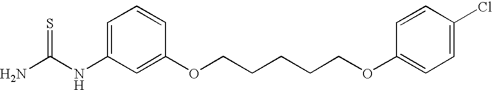 | {3-[5-(4-Chloro- phenoxy)-pentyloxy]- phenyl}-thiourea | 365 |
| 86 | 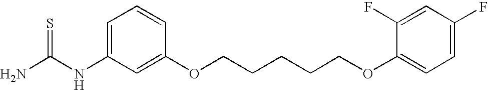 | {3-[5-(2,4-Difluoro- phenoxy)-pentyloxy]- phenyl}-thiourea | 367 |
| 87 | 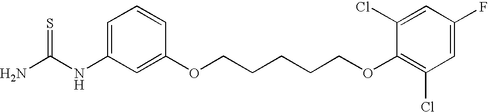 | {3-[5-(2,6-Dichloro-4- fluoro-phenoxy)- pentyloxy]-phenyl}- thiourea | 418 |
| 88 | 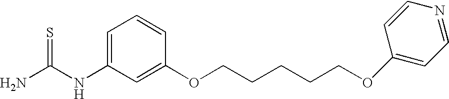 | {3-[5-(Pyridin-4- yloxy)-pentyloxy]- phenyl}-thiourea | 332 |
| 89 | 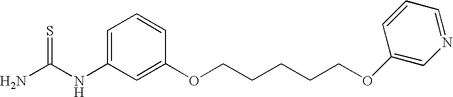 | {3-[5-(Pyridin-3- yloxy)-pentyloxy]- phenyl}-thiourea | 332 |
| 90 | 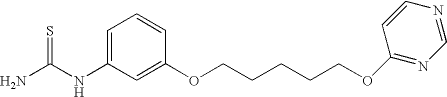 | {3-[5-(Pyrimidin-4- yloxy)-pentyloxy]- phenyl}-thiourea | 333 |
| 91 | 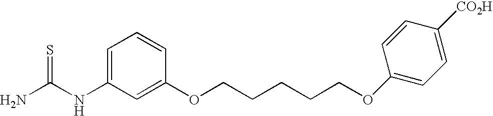 | 4-[5-(3-Thioureido- phenoxy)-pentyloxy]- benzoic acid | 375 |
| 92 | 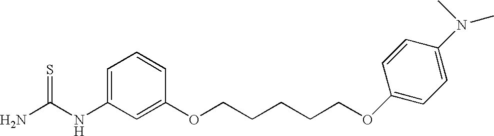 | {3-[5-(4- Dimethylamino- phenoxy)-pentyloxy]- phenyl}-thiourea | 374 |
| 93 | 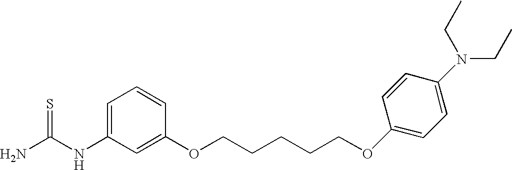 | {3-[5-(4-Diethylamino- phenoxy)-pentyloxy]- phenyl}-thiourea | 402 |
| 94 | 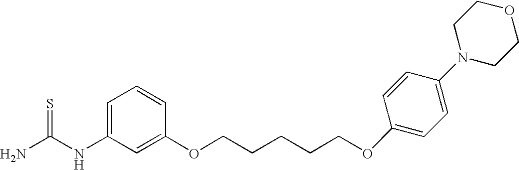 | {3-[5-(4-Morpholin-4- yl-phenoxy)- pentyloxy]-phenyl}- thiourea | 416 |
| 95 | 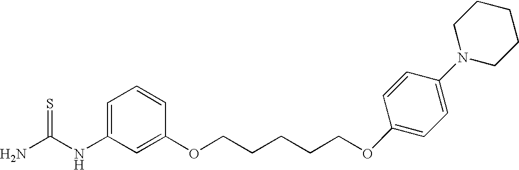 | {3-[5-(4-Piperidin-1- yl-phenoxy)- pentyloxy]-phenyl}- thiourea | 414 |
| 96 | 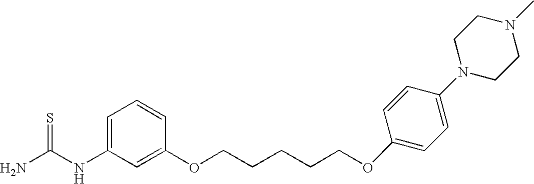 | (3-{5-[4-(4-Methyl- piperazin-1-yl)- phenoxy]-pentyloxy}- phenyl)-thiourea | 429 |
| 97 | 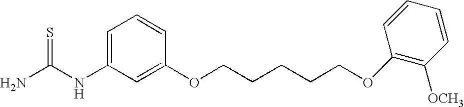 | {3-[5-(2-Methoxy- phenoxy)-pentyloxy]- phenyl}-thiourea | 361 |
| 98 | 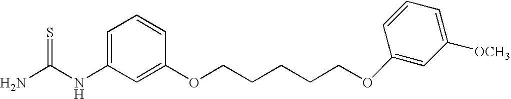 | {3-[5-(3-Methoxy- phenoxy)-pentyloxy]- phenyl}-thiourea | 361 |
| 99 | 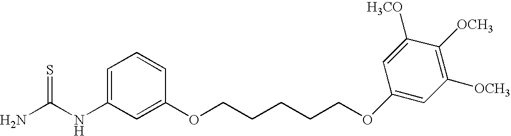 | {3-[5-(3,4,5- Trimethoxy-phenoxy)- pentyloxy]-phenyl}- thiourea | 421 |
| 100 | 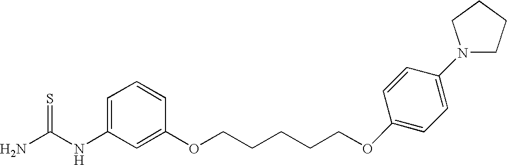 | {3-[5-(4-Pyrrolidin-1- yl-phenoxy)- pentyloxy]-phenyl}- thiourea | 400 |
| 101 | 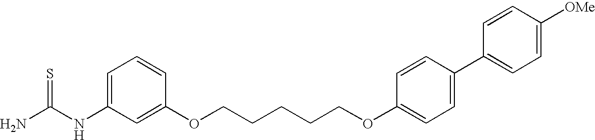 | {3-[5-(4′-Methoxy- biphenyl-4-yloxy)- pentyloxy]-phenyl}- thiourea | 437 |
| 102 | 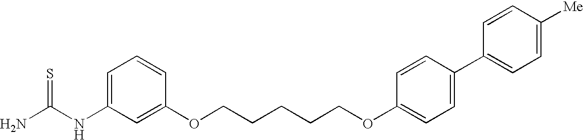 | {3-[5-(4′-Methyl- biphenyl-4-yloxy)- pentyloxy]-phenyl}- thiourea | 421 |
| 103 | 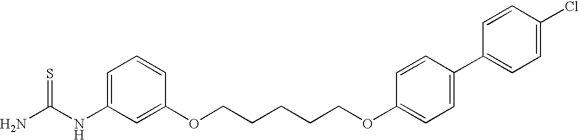 | {3-[5-(4′-Chloro- biphenyl-4-yloxy)- pentyloxy]-phenyl}- thiourea | 441 |
| 104 | 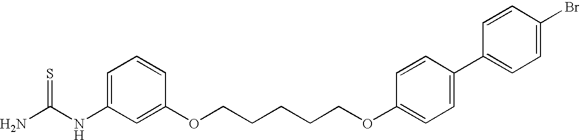 | {3-[5-(4′-Bromo- biphenyl-4-yloxy)- pentyloxy]-phenyl}- thiourea | 485 487 |
| 105 | 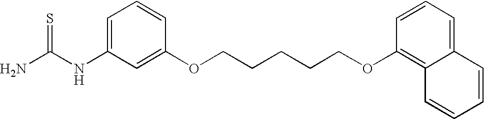 | {3-[5-(Naphthalen-1- yloxy)-pentyloxy]- phenyl}-thiourea | 381 |
| 106 | 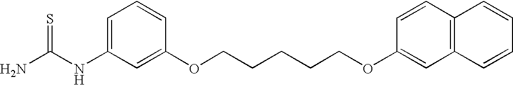 | {3-[5-(Naphthalen-2- yloxy)-pentyloxy]- phenyl}-thiourea | 381 |
| 107 | 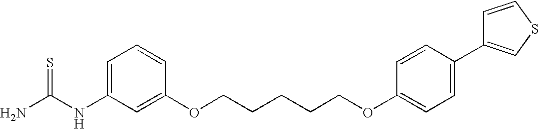 | {3-[5-(4-Thiophen-3- yl-phenoxy)- pentyloxy]-phenyl}- thiourea | 413 |
| 108 | 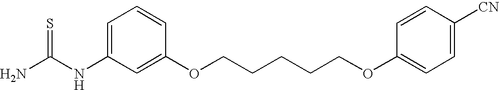 | {3-[5-(4-Cyano- phenoxy)-pentyloxy]- phenyl}-thiourea | 356 |
| 109 | 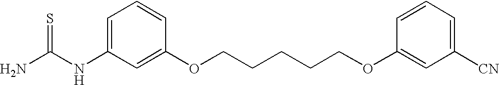 | {3-[5-(3-Cyano- phenoxy)-pentyloxy]- phenyl}-thiourea | 356 |
| 110 | 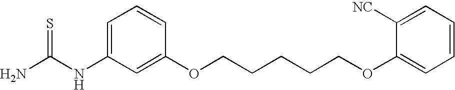 | {3-[5-(2-Cyano- phenoxy)-pentyloxy]- phenyl}-thiourea | 356 |
| 111 | 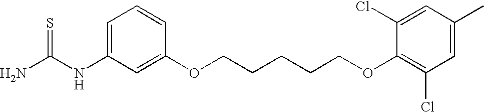 | {3-[5-(2,6-Dichloro-4- methyl-phenoxy)- pentyloxy]-phenyl}- thiourea | 414 |
| 112 | 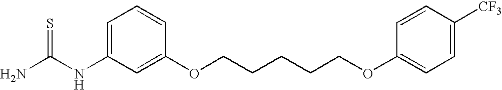 | {3-[5-(4- Trifluoromethyl- phenoxy)-pentyloxy]- phenyl}-thiourea | 399 |
| 113 | 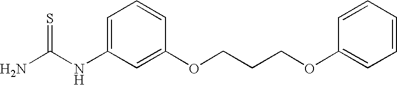 | [3-(3-Phenoxy- propoxy)-phenyl]- thiourea | 303 |
| 114 | 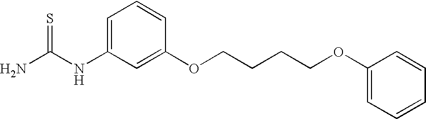 | [3-(4-Phenoxy- butoxy)-phenyl]- thiourea | 317 |
| 115 | 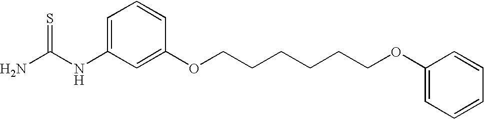 | [3-(6-Phenoxy- hexyloxy)-phenyl]- thiourea | 345 |
| 116 | 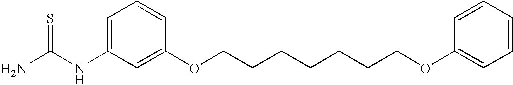 | [3-(7-Phenoxy- heptyloxy)-phenyl]- thiourea | 359 |
| 117 | 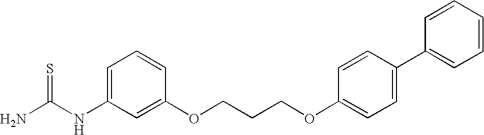 | {4-[3-(Biphenyl-4- yloxy)-propoxy]- phenyl}-thiourea | 379 |
| 118 | 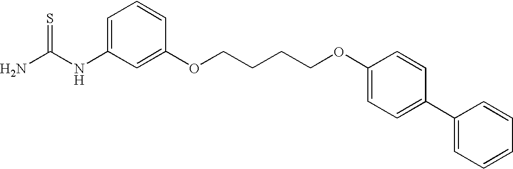 | {3-[4-(Biphenyl-4- yloxy)-butoxy]- phenyl}-thiourea | 393 |
| 119 | 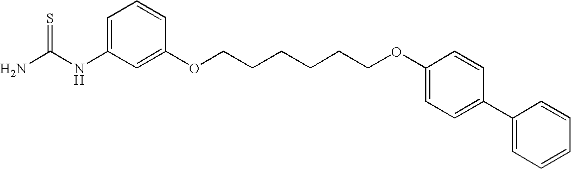 | {3-[6-(Biphenbyl-4- yloxy)-hexyloxy]- phenyl}-thiourea | 421 |
| 120 | 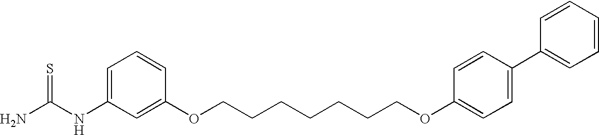 | {3-[7-(Biphenyl-4- yloxy)-heptyloxy]- phenyl}-thiourea | 435 |
| 121 | 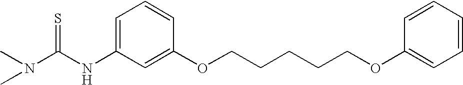 | 1,1-Dimethyl-3-[3-(5- phenoxy-pentyloxy)- phenyl]-thiourea | 359 |
| 122 | 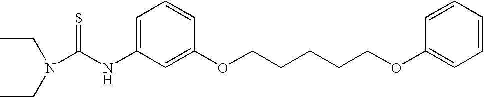 | 1,1-Diethyl-3-[3-(5- phenoxy-pentyloxy)- phenyl]-thiourea | 387 |
| 123 | 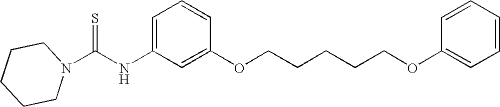 | Piperidine-1- carbothioic acid [3-(5- phenoxy-pentyloxy)- phenyl]-amide | 399 |
| 124 | 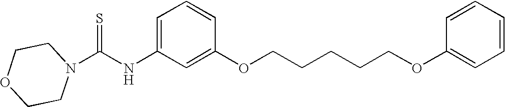 | Morpholine-4- carbothioic acid [3-(5- phenoxy-pentyloxy)- phenyl]-amide | 401 |
| 125 | 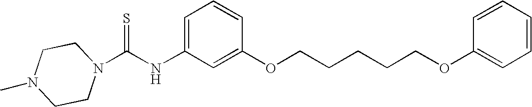 | 4-Methyl-piperazine-1- carbothioic acid [3-(5- phenoxy-pentyloxy)- phenyl]-amide | 414 |
| 126 | 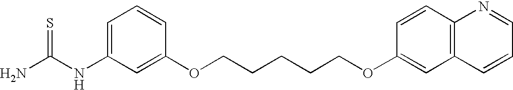 | {3-[5-(Quinolin-6- yloxy)-pentyloxy]- phenyl}-thiourea | 382 |
| 127 | 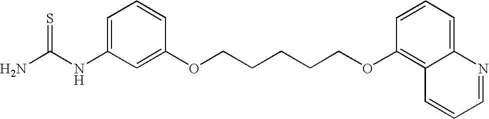 | {3-[5-(Quinolin-5- yloxy)-pentyloxy]- phenyl}-thiourea | 382 |
| 128 | 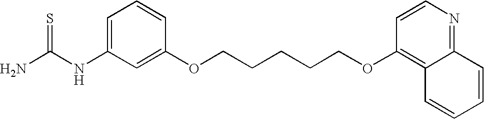 | {3-[5-(Quinolin-4- yloxy)-pentyloxy]- phenyl}-thiourea | 382 |
| 129 | 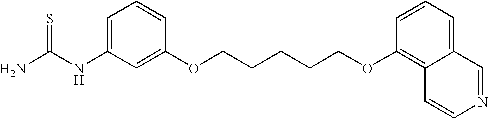 | {3-[5-(Isoquinolin-5- yloxy)-pentyloxy]- phenyl}-thiourea | 382 |
| 130 | 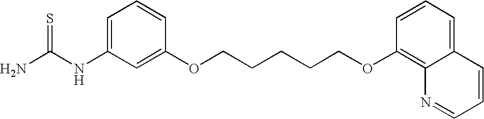 | {3-[5-(Quinolin-8- yloxy)-pentyloxy]- phenyl}-thiourea | 382 |
| 131 | 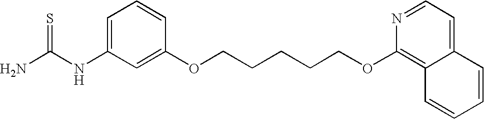 | {3-[5-(Isoquinolin-1- yloxy)-pentyloxy]- phenyl}-thiourea | 382 |
| 132 | 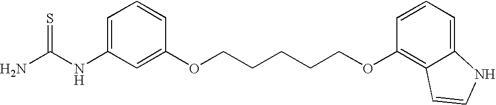 | {3-[5-(1H-Indol-4- yloxy)-pentyloxy]- phenyl}-thiourea | 370 |
| 133 | 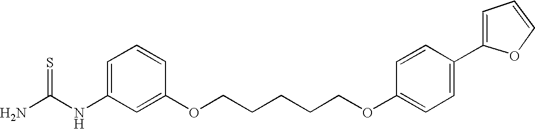 | {3-[5-(4-Furan-2-yl- phenoxy)-pentyloxy]- phenyl}-thiourea | 397 |
| 134 | 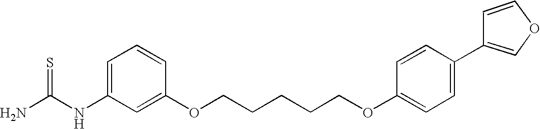 | {3-[5-(4-Furan-3-yl- phenoxy)-pentyloxy]- phenyl}-thiourea | 397 |
| 135 | 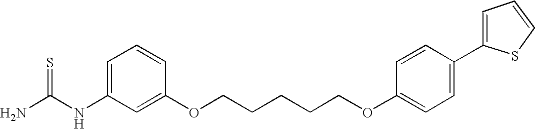 | {3-[5-(4-Thiophen-2- yl-phenoxy)- pentyloxy]-phenyl}- thiourea | 413 |
| 136 | 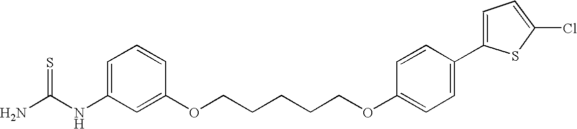 | (3-{5-[4-(5-Chloro- thiophen-2-yl)- phenoxy]-pentyloxy}- phenyl)-thiourea | 447 |
| 137 | 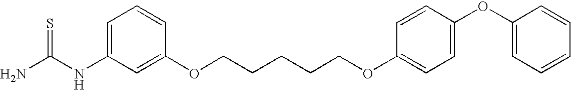 | {3-[5-(4-Phenoxy- phenoxy)-pentyloxy]- phenyl}-thiourea | 423 |
| 138 | 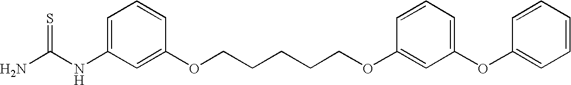 | {3-[5-(3-Phenoxy- phenoxy)-pentyloxy]- phenyl}-thiourea | 423 |
| 139 | 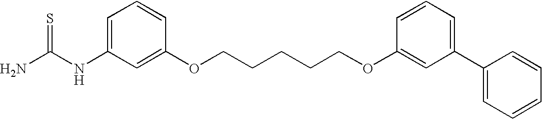 | {3-[5-(Biphenyl-3- yloxy)-pentyloxy]- phenyl}-thiourea | 407 |
| 140 | 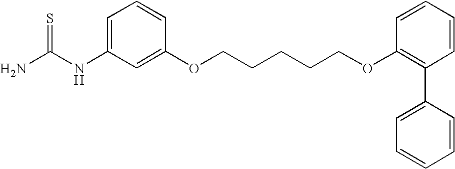 | {3-[5-(Biphenyl-2- yloxy)-pentyloxy]- phenyl}-thiourea | 407 |
| 141 | 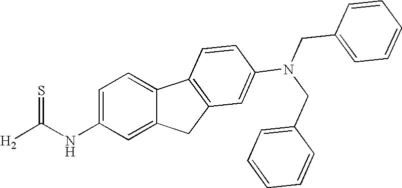 | (7-Dibenzylamino-9H- fluoren-2-yl)-thiourea | 436 |
| 142 | 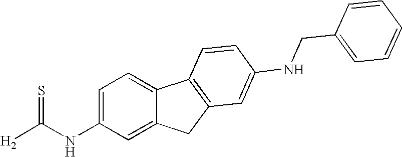 | (7-Benzylamino-9H- fluoren-2-yl)-thiuorea | 346 |
| 143 | 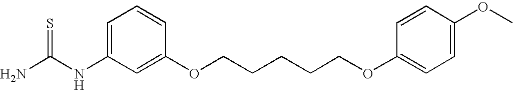 | {3-[5-(4-Methoxy- phenoxy)-pentyloxy]- phenyl}-thiourea | 361 |
| 144 | 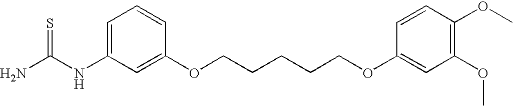 | {3-[5-(3,4-Dimethoxy- phenoxy)-pentyloxy]- phenyl}-thiourea | 391 |
| 145 | 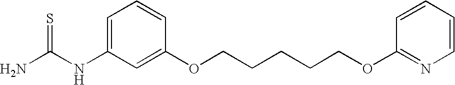 | {3-[5-(Pyridin-2- yloxy)-pentyloxy]- phenyl}-thiourea | 332 |
| 146 | 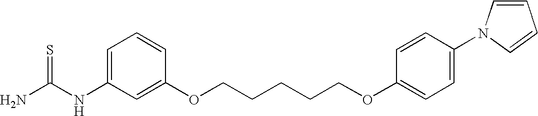 | {3-[5-(4-Pyrrol-1-yl- phenoxy)-pentyloxy]- phenyl}-thiourea | 396 |
| 147 | 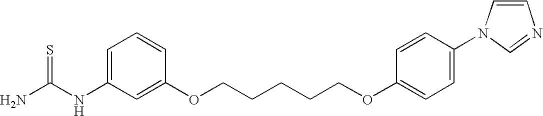 | {3-[5-(4-Imidazol-1-yl- phenoxy)-pentyloxy]- phenyl}-thiourea | 397 |
| 148 | 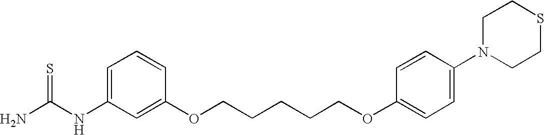 | {3-[5-(4- Thiomorpholin-4-yl- phenoxy)-pentyloxy]- phenyl}-thiourea | 432 |
| 149 | 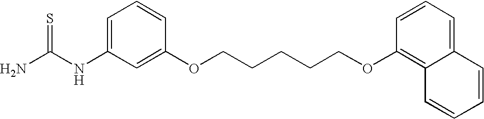 | {3-[7-(Naphthalen-1- yloxy)-heptyloxy]- phenyl}-thiourea | 409 |
| 150 | 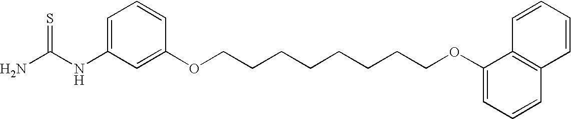 | {3-[8-(Naphthalen-1- yloxy)-octyloxy]- phenyl}-thiourea | 423 |
| 151 | 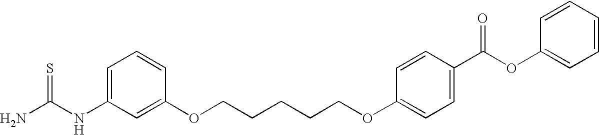 | 4-[5-(3-Thioureido- phenoxy)-pentyloxy]- benzoic acid phenyl ester | 451 |
| 152 | 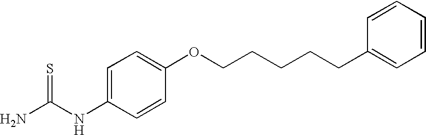 | [4-(5-Phenyl- pentyloxy)-phenyl]- thiourea | 315 |
| 153 | 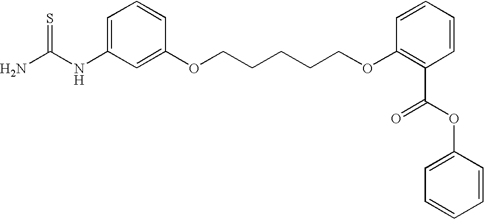 | 2-[5-(3-Thioureido- phenoxy)-pentyloxy]- benzoic acid phenyl ester | 451 |
| 154 | 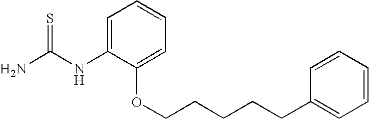 | [2-(5-Phenyl- pentyloxy)-phenyl]- thiourea | 315 |
| 155 | 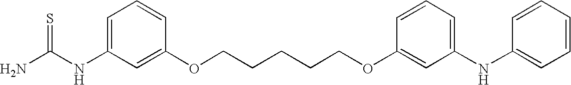 | {3-[5-(3-Phenylamino- phenoxy)-pentyloxy]- phenyl}-thiourea | 422 |
| 156 | 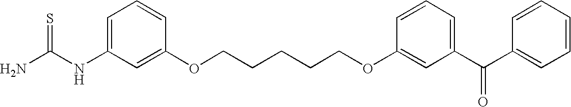 | {3-[5-(3-Benzoyl- phenoxy)-pentyloxy]- phenyl}-thiourea | 435 |
| 157 | 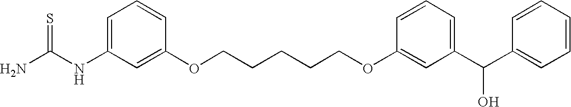 | (3-{5-[3-(Hydroxy- phenyl-methyl)- phenoxy]-pentyloxy}- phenyl)-thiourea | 437 |
| 158 | 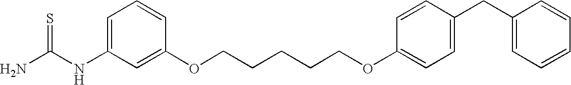 | {3-[5-(4-Benzyl- phenoxy)-pentyloxy]- phenyl}-thiourea | 421 |
| 159 | 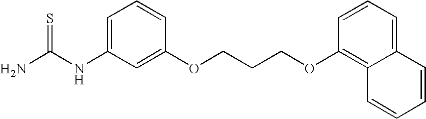 | {3-[3-(Naphthalen-1- yloxy)-propoxy]- phenyl}-thiourea | 353 |
| 160 | 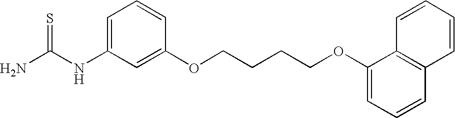 | {3-[4-(Naphthalen-1- yloxy)-butoxy]- phenyl}-thiourea | 367 |
| 161 | 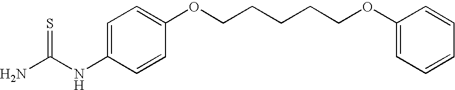 | [4-(5-Phenoxy- pentyloxy)-phenyl]- thiourea | 331 |
| 162 | 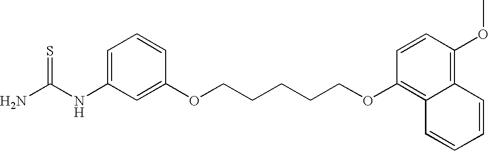 | {3-[5-(4-Methoxy- naphthalen-1-yloxy)- pentyloxy]-phenyl}- thiourea | 411 |
| 163 | 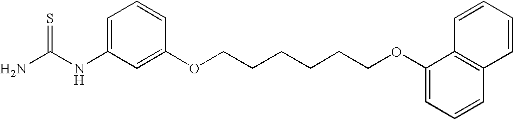 | {3-[6-(Naphthalen-1- yloxy)-hexyloxy]- phenyl}-thiourea | 395 |
| 164 | 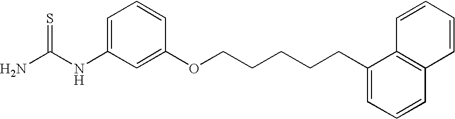 | [3-(5-Naphthalen-1-yl- pentyloxy)-phenyl]- thiourea | 365 |
| 165 | 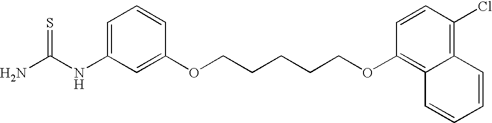 | {3-[5-(4-Chloro- naphthalen-1-yloxy)- pentyloxy]-phenyl}- thiourea | 415 |
| 166 | 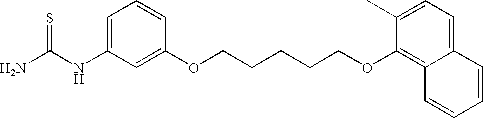 | {3-[5-(2-Methyl- naphthalen-1-yloxy)- pentyloxy]-phenyl}- thiourea | 395 |
| 167 | 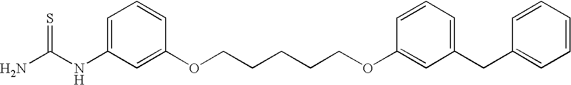 | {3-[5-(3-Benzyl- phenoxy)-pentyloxy]- phenyl}-thiourea | 421 |
| 168 | 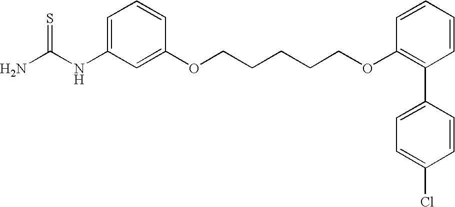 | {3-[5-(4′-Chloro- biphenyl-2-yloxy)- pentyloxy]-phenyl}- thiourea | 441 |
| 169 | 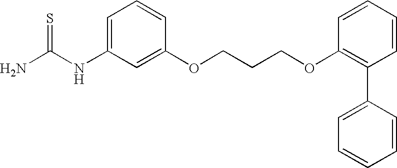 | {3-[3-(Biphenyl-2- yloxy)-propoxy]- phenyl}-thiourea | 379 |
| 170 | 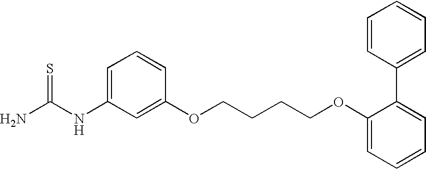 | {3-[4-(Biphenyl-2- yloxy)-butoxy]- phenyl}-thiourea | 393 |
| 171 | 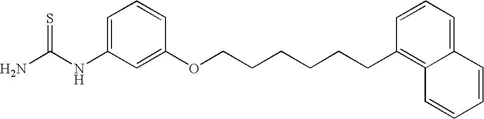 | [3-(6-Naphthalen-1-yl- hexyloxy)-phenyl]- thiourea | 379 |
| 172 | 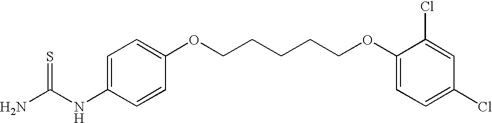 | {4-[5-(2,4-Dichloro- phenoxy)-pentyloxy]- phenyl}-thiourea | 340 |
| 173 | 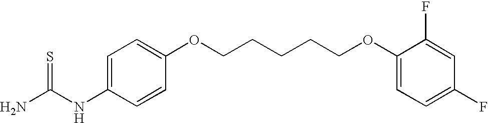 | {4-[5-(2,4-Difluoro- phenoxy)-pentyloxy]- phenyl}-thiourea | 367 |
| 174 | 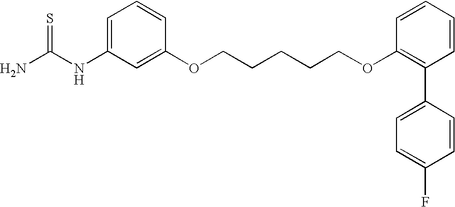 | {3-[5-(4′-Fluoro- biphenyl-2-yloxy)- pentyloxy]-phenyl}- thiourea | 425 |
| 175 | 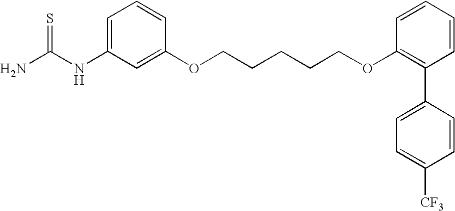 | {3-[5-(4′- Trifluoromethyl- biphenyl-2-yloxy)- pentyloxy]-phenyl}- thiourea | 475 |
| 176 | 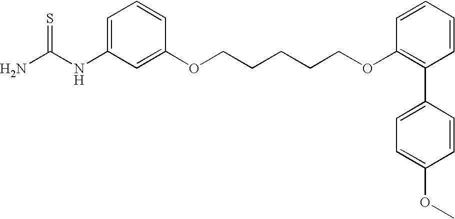 | {3-[5-(4′-Methoxy- biphenyl-2-yloxy)- pentyloxy]-phenyl}- thiourea | 437 |
| 177 | 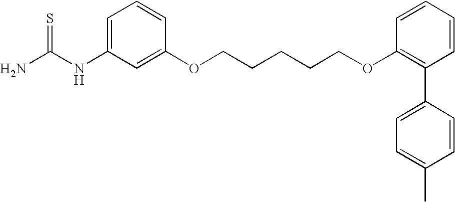 | {3-[5-(4′-Methyl- biphenyl-2-yloxy)- pentyloxy]-phenyl}- thiourea | 421 |
| 178 | 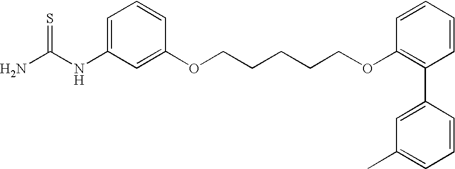 | {3-[5-(3′-Methyl- biphenyl-2-yloxy)- pentyloxy]-phenyl}- thiourea | 421 |
| 179 | 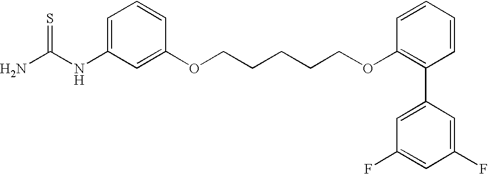 | {3-[5-(3′,5′-Difluoro- biphenyl-2-yloxy)- pentyloxy]-phenyl}- thiourea | 443 |
| 180 | 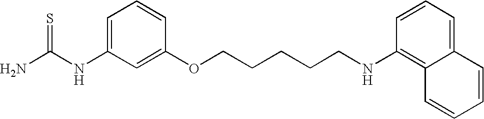 | {3-[5-(Naphthalen-1- ylamino)-pentyloxy]- phenyl}-thiourea | 380 |
| 181 | 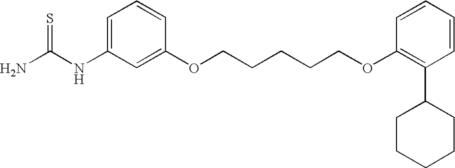 | {3-[5-(2-Cyclohexyl- phenoxy)-pentyloxy]- phenyl}-thiourea | 413 |
| 182 | 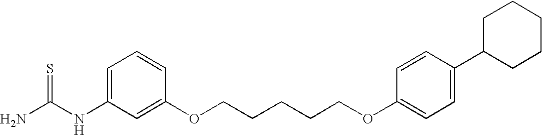 | {3-[5-(4-Cyclohexyl- phenoxy)-pentyloxy]- phenyl}-thiourea | 413 |
| 183 | 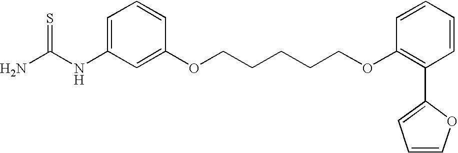 | {3-[5-(2-Furan-2-yl- phenoxy)-pentyloxy]- phenyl}-thiourea | 397 |

The thiourea compounds described above can be prepared by methods well known in the art, Examples 1-183 below provide detailed descriptions of the preparation of compounds 1-183.

Scheme I shown below depicts a typical route for synthesizing certain compounds of the invention. Specifically, 3-nitrophenol can first react, with a brominated aromatic compound via a substitution reaction to form an alkoxy-containing compound. The alkoxy-containing compound can then be reduced, (e.g., by hydrogen or tin chloride) to convert the nitro group to an amino group. The compound thus formed can then be treated with thiocarbonyl diimidazole (TCDI) and a base (e.g., ammonia) to form a compound of the invention (e.g., compounds 1-14, 21-31, 82-140, and 143-183).

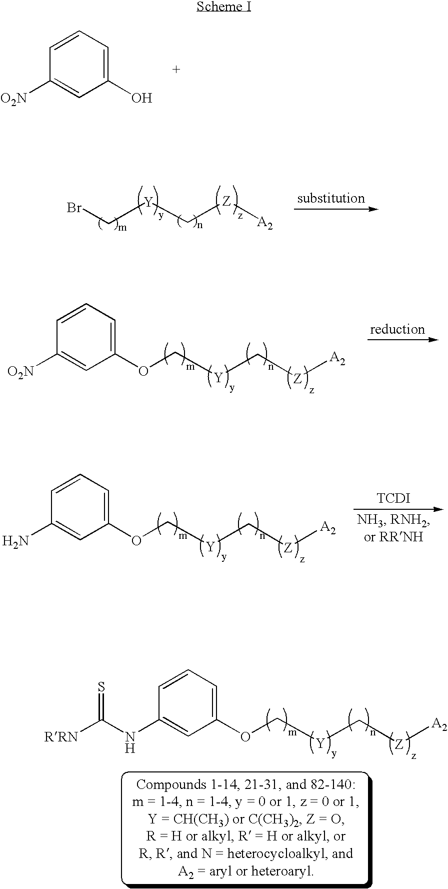

Certain other compounds of the invention can be prepared from benzene-1,3-diamine. For example, as shown in Scheme II below, one of the amino groups on benzene-1,3-diamine can be first protected with a tert-butyloxycarbonyl (BOC) protecting group. The other amino group on benzene-1,3-diamine can then react with a brominated aromatic compound. The compound thus formed can subsequently be deprotected and then treated with thiocarbonyl diimidazole and a base to form compounds of the invention such as compounds 15-20.

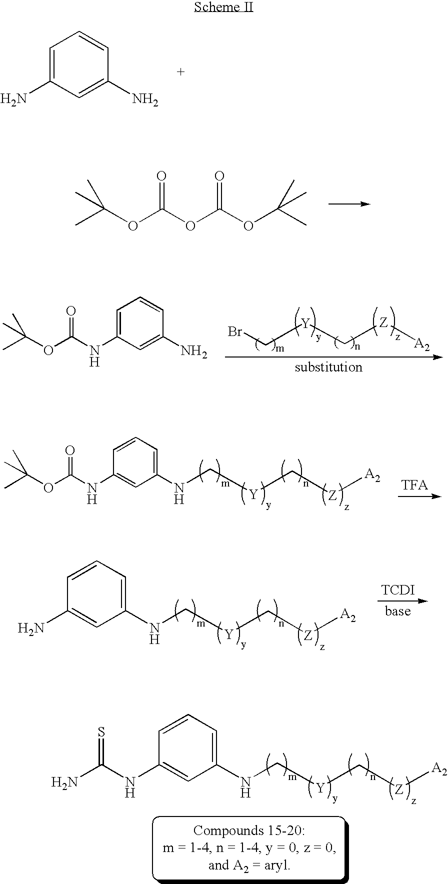

Certain other compounds of the invention can be prepared from a monoamine aromatic compound. For example, as shown in Scheme III below, a monoamino aromatic compound can react with thiocarbonyl diimidazole, followed by ammonia or a primary amine, to form a compound of the invention (e.g., compounds 32-38 and 50-71).

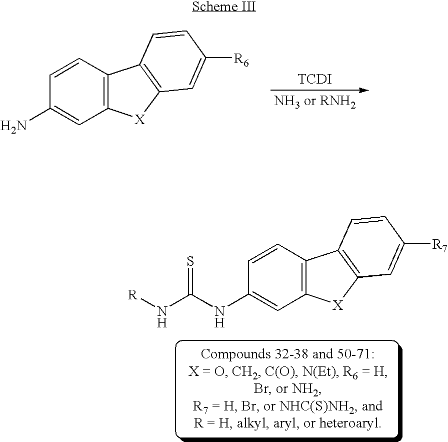

Certain other compounds of the invention can be prepared from a diamine aromatic compound. For example, as shown in Scheme IV below, one amino group on 9H-fluorene-2,7-diamine can first be protected with a BOC protecting group. The other amino group 9H-fluorene-2,7-diamine can then react with a halo-containing compound to form either a compound containing a secondary amino group or a compound containing a tertiary amino group. The compound thus formed can be deprotected (e.g., by reacting with trifluoroacetic acid) and then treated with thiocarbonyl diimidazole and a base to form a compound of the invention (e.g., compounds 39-48, 72-75, 141, and 142).

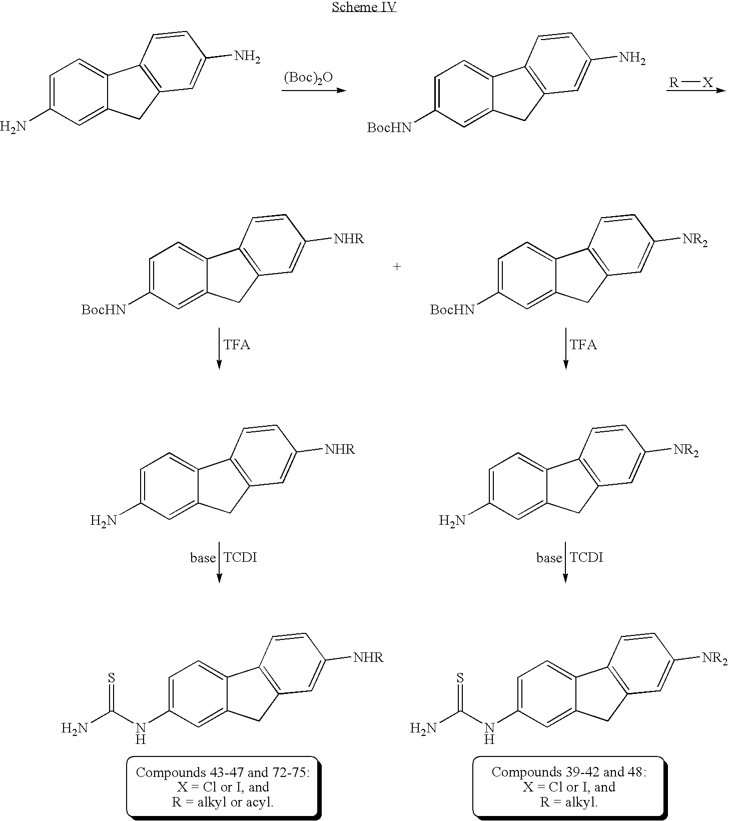

Certain other compounds of the invention containing an imidazolidinyl ring can be prepared by the method shown in Scheme V. Specifically, an amino-containing compound can first react with 1-chloro-2-isothiocyanatoethane to form a chlorine-containing thiourea compound. The thiourea compound can then react with a base (e.g., triethylamine) to form a compound of the invention containing an imidazolidinyl ring (e.g., compounds 76 and 79). The compound thus formed can optionally react with a halo-containing compound to form another compound of the invention (e.g., compounds 77, 78, 80, and 81).

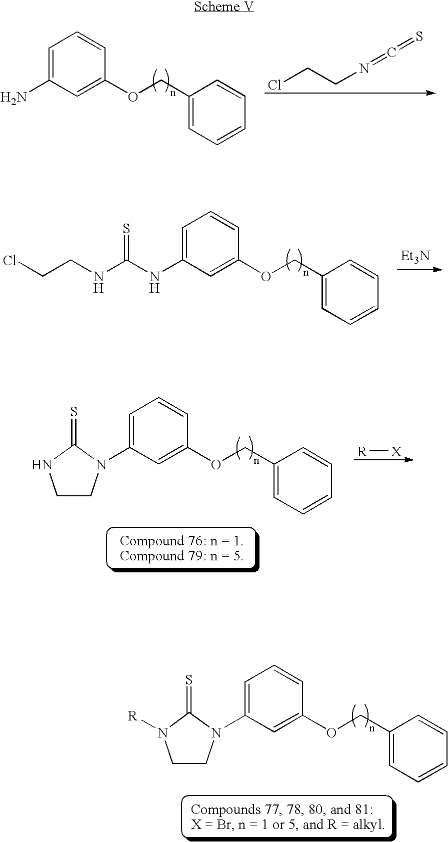

A thiourea compound synthesized above can be purified by a suitable method such as column chromatography, high-pressure liquid chromatography, or recrystallization.

Other thiourea compounds can be prepared using other suitable starting materials through the above synthetic routes and others known in the art. The methods described above may also additionally include steps, either before or after the steps described specifically herein, to add or remove suitable protecting groups in order to ultimately allow synthesis of the thiourea compounds. In addition, various synthetic steps may be performed in an alternate sequence or order to give the desired compounds. Synthetic chemistry transformations and protecting group methodologies (protection and deprotection) useful in synthesizing applicable thiourea compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The thiourea compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition containing an effective amount, of at least one thiourea compound described above and a pharmaceutical acceptable carrier. Further, this invention covers a method of administering an effective amount of one or more of the thiourea compounds to a patient having hepatitis C virus infection, “An effective amount” refers to the amount of an active thiourea compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more thiourea compounds can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water. Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation, for example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active thiourea compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active thiourea compound. Examples of other carriers include colloidal silicon oxide, magnesium stearate, cellulose, sodium lauryl sulfate, and D&C Yellow #10.

The thiourea compounds described above can be preliminarily screened for their efficacy in treating hepatitis C virus infection by an in vitro assay (See Examples 141 and 142 below) and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation, of Compound 1: 1-(3-(5-phenylpentyloxy)phenyl)thiourea

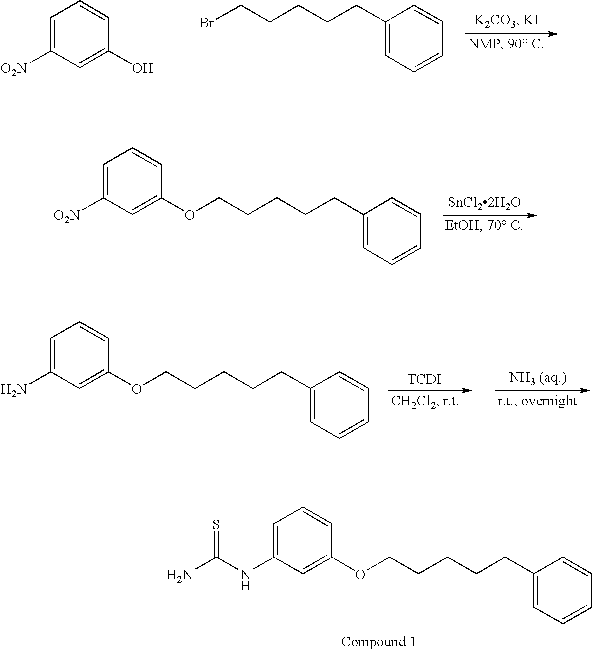

Potassium carbonate (1.2 g, 8.7 mmol) was added to a stirred suspension of 3-nitrophenol (0.8 g, 5.8 mmol), (5-bromo-pentyl)-benzene (1.32 g, 5.8 mmol), and potassium iodide (0.96 g, 5.8 mmol) in N-methylpyrolidinone (15 mL). The mixture was stirred at 90° C. for 4 hours. After the reaction mixture was cooled to the room temperature, it was quenched with water (30 mL) followed, by extraction with ethyl acetate (30 mL×3). The organic layers were combined, washed with brine, and concentrated under vacuum. The residue thus obtained was subjected to column chromatography on silica gel to give 1-nitro-3-(5-phenylpentoxy)benzene (1.4 g, 4.93 mmol, yield: 85%) as colorless oil.

Tin (II) chloride (5.57 g, 24.7 mmol) was added to a solution of 1-nitro-3-(5-phenylpentoxy)benzene (1.4 g, 4.93 mmol) in 35 mL ethanol. The reaction mixture was stirred at 70° C. for 2 hours. After the reaction mixture was cooled to room temperature, a saturated sodium bicarbonate aqueous solution (50 mL) was added. The resultant mixture was extracted with ethyl acetate (2×50 mL). The combined organic phases were washed with brine, dried over anhydrous MgSO₄, and concentrated to give a crude product as a white solid. The crude product was purified by silica gel column chromatography eluting with ethyl acetate-n-hexane to give 3-(5-phenyl-pentyloxy)-phenylamine (1.03 g, 4.04 mmol, yield: 82%) as a white solid.

A solution of 3-(5-phenyl-pentyloxy)phenylamine (200 mg, 1.02 mmol) and thiocarbonyl diimidazole (TCDI, 1.90 mg, 1.06 mmol) in dichloromethane (10 mL) was stirred at room temperature for 2 hours. After a 25% aqueous ammonia solution (2.0 mL, excess) was added, the reaction mixture was stirred at room temperature overnight. The solvent was removed and then the residue thus obtained was purified by silica gel column chromatography eluting with methanol-dichloromethane to give [3-(5-phenyl-pentyloxy)-phenyl]-thiourea (compound 1) (273 mg, (0.87 mmol, yield: 85%) as a white solid.

EI-MS (M+1): 315.

### EXAMPLE 2

### Preparation of Compound 2: 1-(3-(4-phenylbutoxy)phenyl)thiourea

Compound 2 was prepared in a manner similar to that described in Example 1.

EI-MS (M+1): 301.

### EXAMPLE 3

### Preparation of Compound 3: 1-(3-(3-phenylpropoxy)phenyl)thiourea

Compound 3 was prepared in a manner similar to that described in Example 1.

EI-MS (M+1): 287.

### EXAMPLE 4

### Preparation of Compound 4: 1-(3-(6-phenylhexyloxy)phenyl)thiourea

Compound 4 was prepared in a manner similar to that described in Example 1.

EI-MS (M+1): 329.

### EXAMPLE 5

### Preparation of Compound 5: 1-(3-(7-phenylheptyloxy)phenyl)thiourea

Compound 5 was prepared in a manner similar to that described in Example 1.

EI-MS (M+1): 343.

### EXAMPLE 6

### Preparation of Compound 6: 1-(3-(8-phenyloctyloxy)phenyl)thiourea

Compound 6 was prepared in a manner similar to that described in Example 1.

EI-MS (M+1): 357.

### EXAMPLE 7

### Preparation of Compound 7: 1-(3-(5-phenoxypentyloxy)phenyl)thiourea

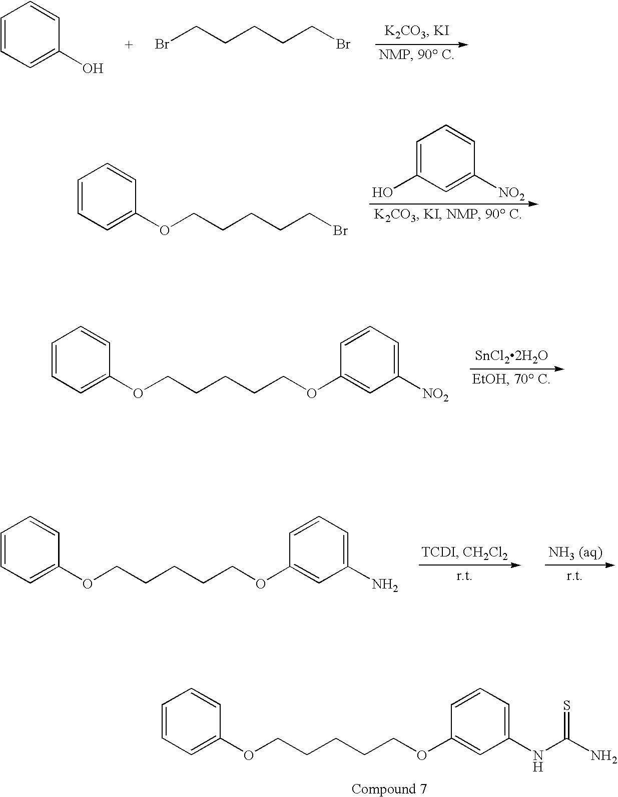

Potassium carbonate (10.35 g, 75.0 mmol) was added to a stirred suspension of phenol (4.7 g, 50.0 mmol), 1,5-dibromopentane (12.65 g, 55.0 mmol), and potassium iodide (0.83 g, 5.0 mmol) m N-methylpyrrolidinone (100 mL). The reaction mixture was stirred at 90° C. for 4 hours. It was then quenched with water (30 mL), followed by extraction with ethyl acetate (30 mL×3). The organic layers were combined, washed with brine, and concentrated under vacuum. The residue thus obtained was subjected to column chromatography on silica gel to give (5-bromopentyloxy)benzene (12.0 g, 49.38 mmol, yield: 98%) as yellow oil.

Potassium carbonate (10.35 g, 75.0 mmol) was added to a stirred suspension of (5-bromopentyloxy)benzene (12.0 g, 49.38 mmol), 3-nitrophenol (6.95 g, 50.0 mmol), and potassium iodide (0.83 g, 5.0 mmol) in N-methylpyrolidinone (100 mL). The reaction mixture was stirred at 90° C. for 4 hours. It was then quenched with water (30 mL), followed by extraction with ethyl acetate (30 ml×3). The organic layers were combined, washed with brine, and concentrated under vacuum. The residue thus obtained was subjected to column chromatography on silica gel to give 1-nitro-3-(5-phenoxypentoxy)benzene (11.89 g, 39.5 mmol, yield: 80%) as colorless oil.

Tin (II) chloride (19.78 g, 87.89 mmol) was added to a solution of 1-nitro-3-(5-phenoxypentoxy)benzene (5.29 g, 17.58 mmol) in 100 mL ethanol. The reaction mixture was stirred at 70° C. for 2 hours. After the reaction mixture was cooled to room temperature, a saturated sodium bicarbonate aqueous solution (50 mL) was added. The solution was extracted with ethyl acetate (3×50 mL), and the combined organic phases were washed with brine, dried over anhydrous MgSO₄, and concentrated to give a crude product as a white solid. The crude product was purified by silica gel column chromatography eluting with ethyl acetate-n-hexane to give 3-(5-phenoxy-pentyloxy)-phenylamine (4.67 g, 17.22 mmol, yield: 98%) as a light yellow solid.

A solution of 3-(5-phenoxy-pentyloxy)phenylamine (200 mg, 0.74 mmol) and thiocarbonyl diimidazole (TCDI, 158 mg, 0.89 mmol) in dichloromethane (3 mL) was stirred at room temperature for 2 hours. After a 25% ammonia aqueous solution (2 mL, excess) was added, the reaction mixture was stirred at room temperature overnight. The solvent was then removed and the residue thus obtained was purified by silica gel column chromatography eluting with methanol-dichloromethane to give [3-(5-phenoxy-pentyloxy)-phenyl]-thiourea (compound 7) (126 mg, 0.38 mmol, yield: 52%) as a white solid.

EI-MS (M+1): 331.

### EXAMPLE 8

### Preparation of Compound 8: ethyl 4-(5-(3-thioureidophenoxy)pentyloxy)-benzoate

Compound 8 was prepared in a manner similar to that described in Example 7.

EI-MS (M+1): 403.

### EXAMPLE 9

### Preparation of Compound 9: 1-(3-(5-(4-bromophenoxy)pentyloxy)phenyl)-thiourea

Compound 9 was prepared in a manner similar to that described in Example 7.

EI-MS (M+1): 409, 411.

### EXAMPLE 10

### Preparation of Compound 10: 1-(3-(3-methyl-5-phenoxypentyloxy)phenyl)-thiourea

Compound 10 was prepared in a manner similar to that described in Example 7.

EI-MS (M+1): 345.

### EXAMPLE 11

### Preparation of Compound 11: 1-(3-(3,3-dimethyl-5-phenoxypentyloxy)-phenyl)thiourea

Compound 11 was prepared in a manner similar to that described in Example 7.

EI-MS (M+1): 359.

### EXAMPLE 12

### Preparation of Compound 12: 1-(3-(5-(biphenyl-4-yloxy)pentyloxy)phenyl)-thiourea

Compound 12 was prepared in a manner similar to that described in Example 7.

EI-MS (M+1): 407.

### EXAMPLE 13

### Preparation of Compound 13: 1-(3-(5-(biphenyl-4-yloxy)-3-methylpentyl-oxy)phenyl) thiourea

Compound 13 was prepared in a manner similar to that described in Example 7.

EI-MS (M+1): 421.

### EXAMPLE 14

### Preparation of Compound 14: 1-(3-(5-(biphenyl-4-yloxy)-3,3-dimethyl-pentyloxy)phenyl)thiourea

Compound 14 was prepared in a manner similar to that described in Example 7.

EI-MS (M+1): 435.

### EXAMPLE 15

### Preparation of Compound 15: 1-(3-(5-phenylpentylamino)phenyl)thiourea

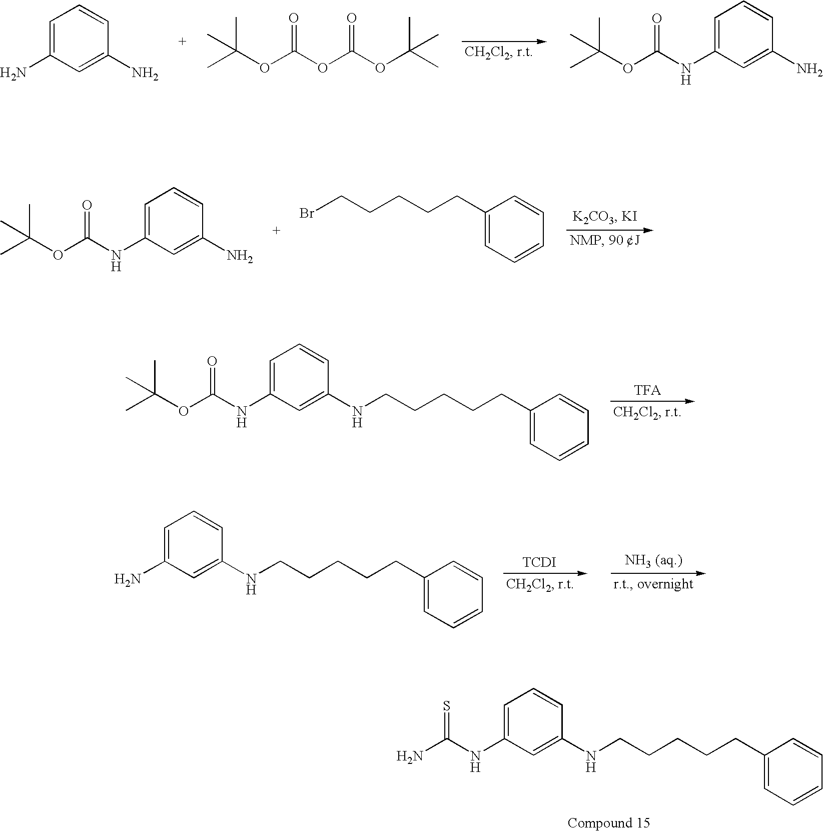

(BOC)₂O (10.1 g, 46.3 mmol) was added to a solution of benzene-1,3-diamine (5.0 g, 46.3 mmol) in dichloromethane (80 mL). The reaction mixture was stirred at room temperature for 60 hours. The reaction mixture was quenched with, water (30 mL), followed by extraction with ethyl acetate (30 mL×3). The organic layers were combined, washed with brine, and concentrated under vacuum. The residue thus obtained was subjected to column chromatography on silica gel to give (3-aminophenyl)-carbamic acid tert-butyl ester (4.34 g, 20.8 mmol, yield: 45%) as a white solid.

Potassium carbonate (0.6 g, 4.35 mmol) was added to a stirred suspension of (3-amino-phenyl)-carbamic acid tert-butyl ester (0.6 g, 2.9 mmol), (5-bromo-pentyl)benzene (0.66 g, 2.9 mmol), and potassium iodide (0.48 g, 2.9 mmol) in N-methylpyrolidinone (14 mL). The reaction mixture was stirred at 90° C. for 4 hours. It was quenched with water (30 mL), followed by extraction with ethyl acetate (30 mL×3). The organic layers were combined, washed with brine, and concentrated under vacuum. The residue thus obtained was subjected to column chromatography on silica gel to give [3-(5-phenyl-pentylamino)-phenyl]-carbamic acid tert-butyl ester (802 mg, 2.26 mmol, yield: 78%) as yellow oil.

Trifluoroacetic acid (TFA, 2.0 mL, 26.3 mmol) was added to a solution of [3-(5-phenyl-pentylamino)-phenyl]-carbamic acid tert-butyl ester (802 mg, 2.26 mmol) in 10 mL dichloromethane. The reaction mixture was stirred at room temperature for 1 hour. It was then quenched with water (30 mL), followed by extraction with ethyl acetate (30 mL×3). The organic layers were combined, washed with brine, and concentrated under vacuum. The residue thus obtained was subjected to column chromatography on silica gel to give N-(5-phenyl-pentyl)-benzene-1,3-diamine (529 mg, 2.08 mmol, yield: 92%) as light yellow solid.

A solution of N-(5-phenyl-pentyl)-benzene-1,3-diamine (89 mg, 0.4 mmol) and thiocarbonyl diimidazole (TCDI, 74 mg, 0.42 mmol) in dichloromethane (4 mL) was stirred at room temperature for 2 hours. After a 25% aqueous ammonia solution (2.0 mL, excess) was added, the reaction mixture was stirred at room temperature overnight. The solvent was then removed and the residue thus obtained was purified by silica gel column chromatography eluting with methanol-dichloromethane to give [3-(5-phenylpentylamino)-phenyl]-4-thiourea (compound 17) (113 mg, 0.36 mmol, yield: 90%) as a white solid.

EI-MS (M+1): 314.

### EXAMPLE 16

### Preparation of Compound 16: 1-(3-(4-phenylbutylamino)phenyl)thiourea

Compound 16 was prepared in a manner similar to that described in Example 15.

EI-MS (M+1): 300.

### EXAMPLE 17

### Preparation of Compound 17: 1-(3-(3-phenylpropylamino)phenyl)thiourea

Compound 17 was prepared in a manner similar to that described in Example 15.

EI-MS (M+1): 286.

### EXAMPLE 18

### Preparation of Compound 18: 1-(3-(6-phenylhexylamino)phenyl)thiourea

Compound 18 was prepared in a manner similar to that described in Example 15.

EI-MS (M+1): 328.

### EXAMPLE 19

### Preparation of Compound 19: 1-(3-(7-phenylheptylamino)phenyl)thiourea

Compound 19 was prepared in a manner similar to that described in Example 15.

EI-MS (M+1): 342.

### EXAMPLE 20

### Preparation of Compound 20: 1-(3-(8-phenyloctylamino)phenyl)thiourea

Compound 20 was prepared in a manner similar to that described in Example 15.

EI-MS (M+1): 356.

### EXAMPLE 21

### Preparation of Compound 21: 1-methyl-3-(3-(5-phenylpentyloxy)phenyl)-thiourea

Compound 21 was prepared in a manner similar to that described in Example 1.

EI-MS (M+1): 329.

### EXAMPLE 22

### Preparation of Compound 22: ethyl-3-(3-(5-phenylpentyloxy)phenyl)-thiourea

Compound 22 was prepared in a manner similar to that described in Example 1.

EI-MS (M+1): 343.

### EXAMPLE 23

### Preparation of Compound 23: 1-(3-(5-phenylpentyloxy)phenyl)-3-propyl-thiourea

Compound 23 was prepared in a manner similar to that described in Example 1.

EI-MS (M+1): 357.

### EXAMPLE 24

### Preparation of Compound 24: 1-butyl-3-(3-(5-phenylpentyloxy)phenyl)-thiourea

Compound 24 was prepared in a manner similar to that described in Example 1.

EI-MS (M+1): 371.

### EXAMPLE 25

### Preparation of Compound 25: 1-pentyl-3-(3-(5-phenylpentyloxy)phenyl)-thiourea

Compound 25 was prepared in a manner similar to that described in Example 1.

EI-MS (M+1): 385.

### EXAMPLE 26

### Preparation of Compound 26: 1-hexyl-3-(3-(5-phenylpentyloxy)phenyl)-thiourea

Compound 26 was prepared in a manner similar to that described in Example 1.

EI-MS (M+1): 399.

### EXAMPLE 27

### Preparation of Compound 27: 1-heptyl-3-(3-(5-phenylpentyloxy)phenyl)-thiourea

Compound 27 was prepared in a manner similar to that described in Example 1.

EI-MS (M+1): 413.

### EXAMPLE 28

### Preparation of Compound 28: 1-octyl-(3-(3-(5-phenylpentyloxy)phenyl)-thiourea

Compound 28 was prepared in a manner similar to that described in Example 1.

EI-MS (M+1): 427.

### EXAMPLE 29

### Preparation of Compound 29: 1-phenethyl-3-(3-(5-phenylpentyloxy)-phenyl)thiourea

Compound 29 was prepared in a manner similar to that described in Example 1.

EI-MS (M+1): 419.

### EXAMPLE 30

### Preparation of Compound 30: 1-(3-(5-phenylpentyloxy)phenyl)-3-(3-phenylpropyl)thiourea

Compound 30 was prepared in a manner similar to that described in Example 1.

EI-MS (M+1): 433.

### EXAMPLE 31

### Preparation of Compound 31: 1-(4-phenylbutyl)-3-(3-(5-phenylpentyloxy)phenyl)thiourea

Compound 31 was prepared in a manner similar to that described in Example 1.

EI-MS (M+1): 447.

### EXAMPLE 32

### Preparation of Compound 32: 1-(7-bromo-9H-fluoren-2-yl)thiourea

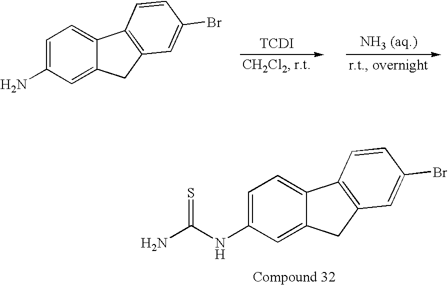

A solution of 7-bromo-9H-fluoren-2-ylamine (0.3 g, 1.0 mmol) and thiocarbonyl diimidazole (TCDI, 0.2 g, 1.2 mmol) in dichloromethane (10 mL) was stirred at room temperature for 2 hours. After a 25% aqueous ammonia solution (2.0 mL, excess) was added, the reaction mixture was stirred at room temperature overnight. The solvent was then removed and the residue thus obtained was purified by silica gel column chromatography eluting with methanol-dichloromethane to give (7-bromo-9H-fluoren-2-yl)-thiourea (compound 32) (297 mg, 0.93 mmol, yield 93%) as a white solid.

EI-MS (M+1): 320.

### EXAMPLE 33

### Preparation of Compound 33: 1-(9-ethyl-9H-carbazol-3-yl)thiourea

Compound 33 was prepared in a manner similar to that described in Example 32.

EI-MS (M+1): 270.

### EXAMPLE 34

### Preparation of Compound 34: 1-(9-oxo-9H-fluoren-2-yl)thiourea

Compound 34 was prepared in a manner similar to that described in Example 32.

EI-MS (M+1): 255.

### EXAMPLE 35

### Preparation of Compound 35: 1-(7-bromo-9-oxo-9H-fluoren-2-yl)thiourea

Compound 35 was prepared in a manner similar to that described in Example 32.

EI-MS (M+1): 332, 334.

### EXAMPLE 36

### Preparation of Compound 36: 1-(9-oxo-9H-fluoren-3-yl)thiourea

Compound 36 was prepared in a manner similar to that described in Example 32.

EI-MS (M+1): 255.

### EXAMPLE 37

### Preparation of Compound 37: 1-(9H-fluoren-2-yl)thiourea

Compound 37 was prepared in a manner similar to that described in Example 32.

EI-MS (M+1): 241.

### EXAMPLE 38

### Preparation of Compound 38: 1-(2-methoxydibenzo[b,d]furan-3-yl)thiourea

Compound 38 was prepared in a manner similar to that described in Example 32.

EI-MS (M+1): 273.

### EXAMPLE 39

### Preparation of Compound 39: 1 (7-(dipropylamino)-9H-fluoren-2-yl)thiourea

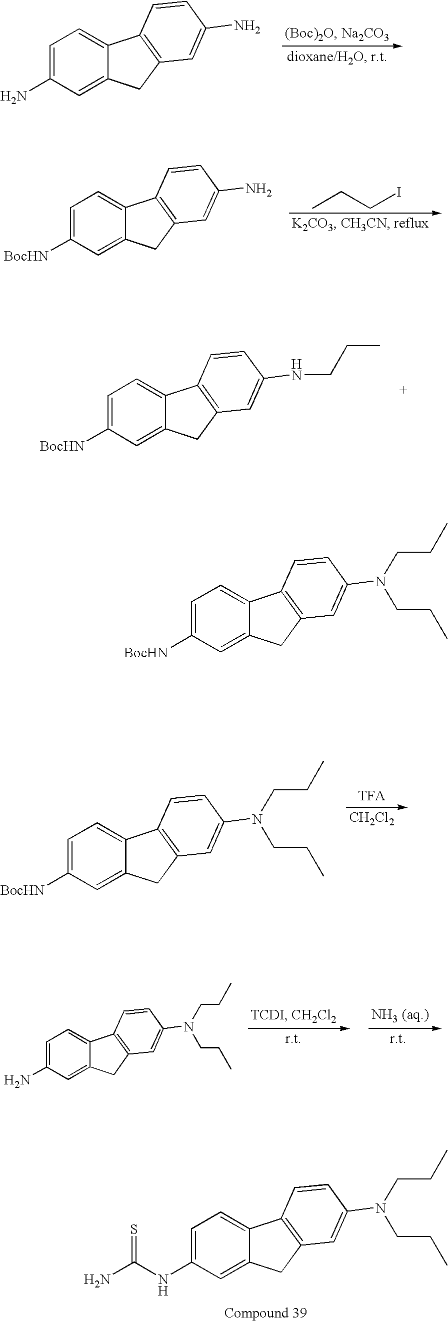

Sodium carbonate (1.06 g, 10.0 mmol) was added to a solution of 9H-fluorene-2,7-diamine (1.0 g, 5.0 mmol) and (BOC)₂O (1.4 mL, 7.5 mmol) in 1,4-dioxane (20 mL) and H₂O (10 mL). The reaction mixture was stirred at room temperature overnight. It was then quenched with saturated ammonium chloride aqueous solution (30 mL), followed by extraction with ethyl acetate (30 mL×3). The organic layers were combined, washed with brine, and concentrated under vacuum. The residue thus obtained was subjected to column chromatography on silica gel to give (7-amino-9H-fluoren-2-yl)-carbamic acid tert-butyl ester (640 mg, 2.16 mmol yield: 43%) as a yellow solid.

Potassium carbonate (120 mg, 0.87 mmol) was added to a stirred suspension of (7-amino-9H-fluoren-2-yl)-carbamic acid tert-butyl ester (200 mg, 0.67 mmol), n-propyl iodide (114 mg, 0.67 mmol) in acetonitrile (20 mL). The reaction mixture was stirred at refluxing temperature for 4 hours. It was then quenched with a saturated ammonium chloride aqueous solution (30 mL), followed by extraction with ethyl acetate (30 mL×3). The organic layers were combined, washed with brine, and concentrated under vacuum. The residue thus obtained was subjected to column chromatography on silica gel to give ((7-propylamino-9H-fluoren-2-yl)-carbamic acid, tert-butyl ester (91 mg, 0.27 mmol, yield: 40%) as a light brown solid and (7-dipropylamino-9H-fluoren-2-yl)carbamic acid tert-butyl ester (114 mg, 0.30 mmol, yield: 45%) as a light brown solid.

Trifluoroacetic acid (TFA, 2.0 mL, 26.3 mmol) was added to a solution of (7-dipropylamino-9H-fluoren-2-yl)-carbamic acid tert-butyl ester (270 mg, 0.71 mmol) in 20 mL dichloromethane. The reaction mixture was stirred at room temperature for 1 hour. It was then quenched with water (30 mL), followed by extraction with ethyl acetate (30 mL×3). The organic Savers were combined, washed with brine, and concentrated under vacuum. The residue thus obtained was subjected to column chromatography on silica gel to give N,N-dipropyl-9H-fluorene-2,7-diamine (220 mg, 0.78 mmol, yield: 91%) as a light brown solid.

A solution of N,N-dipropyl-9H-fluorene-2,7-diamine (220 mg, 0.78 mmol) and thiocarbonyl diimidazole (TCDI, 163 mg, 0.92 mmol) in dichloromethane (5 mL) was stirred at room temperature for 2 hours. After a 25% ammonia aqueous solution (2.0 mL, excess) was added, the reaction mixture was stirred at room temperature overnight. The solvent was then removed and the residue thus obtained was purified by silica gel column chromatography eluting with methanol-dichloromethane to give (7-dipropylamino-9H-fluoren-2-yl)-thiourea (compound 39) (231 mg, 0.69 mmol, yield; 88%) as a white solid.

EI-MS (M+1): 340.

### EXAMPLE 40

### Preparation of Compound 40: 1-(7-(diethylamino)-9H-fluoren-2-yl)thiourea

Compound 40 was prepared in a manner similar to that described in Example 39.

EI-MS (M+1): 312.

### EXAMPLE 41

### Preparation of Compound 41: 1-(7-(dimethylamino)-9H-fluoren-2-yl)thiourea

Compound 41 was prepared in a manner similar to that described in Example 39.

EI-MS (M+1): 284.

### EXAMPLE 42

### Preparation of Compound 42: 1-(7-(dibutylamino)-9H-fluoren-2-yl)thiourea

Compound 42 was prepared in a manner similar to that described in Example 39.

EI-MS (M+1): 368.

### EXAMPLE 43

### Preparation of Compound 43: 1-(7-(propylamino)-9H-fluoren-2-yl)thiourea

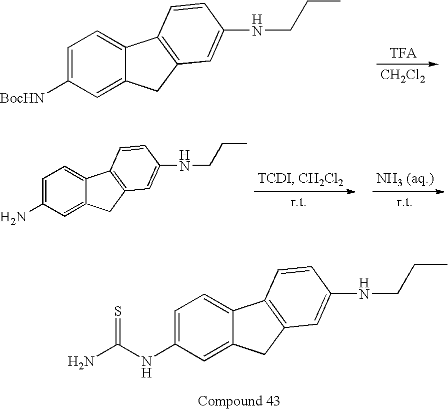

Trifluoroacetic acid (TFA, 2.0 mL, 26.3 mmol) was added to a solution of (7-propylamino-9H-fluoren-2-yl)-carbamic acid tert-butyl ester (91 mg, 0.27 mmol) prepared in Example 39 in 10 mL dichloromethane. The reaction mixture was stirred at room temperature for 1 hour. It was then quenched with water (30 mL), followed by extraction with ethyl acetate (30 mL×3). The organic layers were combined, washed with brine, and concentrated under vacuum. The residue thus obtained was subjected to column chromatography on silica gel to give N²-propyl-9H-fluorene-2,7-diamine (60 mg, 0.25 mmol, yield: 92%) as a light brown solid.

A solution of N²-propyl-9H-fluorene-2,7-diamine (60 mg, 0.25 mmol) and thiocarbonyl diimidazole (53 mg, 0.30 mmol) in dichloromethane (5 mL) was stirred at room temperature for 2 hours. After a 25% ammonia aqueous solution (2.0 mL, excess) was added, the reaction mixture was stirred at room temperature overnight. The solvent was then removed and the residue was purified by silica gel column chromatography eluting with methanol-dichloromethane to give (7-propylamino-9H-fluoren-2-yl)-thiourea (compound 43) (68 mg, 0.23 mmol, yield: 90%) as a white solid.

EI-MS (M+1): 298.

### EXAMPLE 44

### Preparation of Compound 44: 1-(7-(ethylamino)-9H-fluoren-2-yl)thiourea

Compound 44 was prepared in a manner similar to that described in Example 43.

EI-MS (M+1): 284.

### EXAMPLE 45

### Preparation of Compound 45: 1-(7-(methylamino)-9H-fluoren-2-yl)thiourea

Compound 45 was prepared in a manner similar to that described in Example 43.

EI-MS (M+1): 270.

### EXAMPLE 46

### Preparation of Compound 46: 1-(7-(butylamino)-9H-fluoren-2-yl)thiourea

Compound 46 was prepared in a manner similar to that described in Example 43.

EI-MS (M+1): 312.

### EXAMPLE 47

### Preparation of Compound 47: 1-(7-(3-phenylpropylamino)-9H-fluoren-2-yl)thiourea

Compound 47 was prepared in a manner similar to that described in Example 43.

EI-MS (M+1): 374.

### EXAMPLE 48

### Preparation of Compound 48: 1-(7-(bis(3-phenylpropyl)amino)-9H-fluoren-2-yl)thiourea

Compound 48 was prepared in a manner similar to that described in Example 43.

EI-MS (M+1): 492.

### EXAMPLE 49

### Preparation of Compound 49: 1-(7-amino-9H-fluoren-2-yl)thiourea

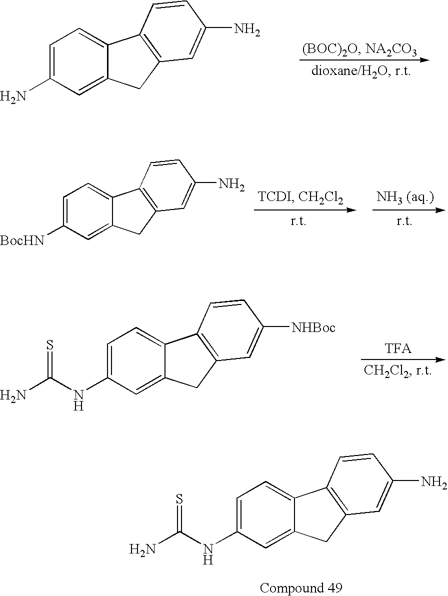

Sodium carbonate (1.06 g, 10.0 mmol) was added to a solution of 9H-fluorene-2,7-diamine (1.0 g, 5.0 mmol) and (BOC)₂O (1.4 mL, 7.5 mmol) in dioxane (20 mL) and H₂O (10 mL) at room temperature. The reaction mixture was stirred at room temperature overnight. It was then quenched with water (30 mL) followed by extraction with ethyl acetate (30 mL×3). The organic layers were combined, washed with brine, and concentrated under vacuum. The residue thus obtained was subjected to column chromatography on silica gel to give (7-amino-9H-fluoren-2-yl)-carbamic acid tert-butyl ester (640 mg, 2.16 mmol, yield: 43%) as a yellow solid.

A solution of (7-amino-9H-fluoren-2-yl)-carbamic acid tert-butyl ester (116 mg, 0.39 mmol) and thiocarbonyl diimidazole (81 mg, 0.45 mmol) in dichloromethane (5 mL) was stirred at room temperature for 2 hours. After a 25% ammonia aqueous solution (2.0 mL, excess) was added, the reaction mixture was stirred at room temperature overnight. The solvent was then removed and the residue thus obtained was purified by silica gel column chromatography eluting with methanol-dichloromethane to give (7-thioureido-9H-fluoren-2-yl)-carbamic acid tert-butyl ester (118 mg, 0.33 mmol, yield: 85%) as a white solid.

Trifluoroacetic acid (TFA, 2.0 mL, 26.3 mmol) was added to a solution of (7-thioureido-9H-fluoren-2-yl)-carbamic acid tert-butyl ester (75 mg, 0.21 mmol) in 2 mL dichloromethane. The reaction mixture was stirred at room temperature for 1 hour. It was then quenched with water (30 mL), followed by extraction with ethyl acetate (30 mL×3). The organic layers were combined, washed with brine, and concentrated under vacuum. The residue thus obtained was subjected to column chromatography on silica gel to give (7-amino-9-1H-fluoren-2-yl)-thiourea (compound 49) (51 mg, 0.20 mmol, yield: 95%) as a white solid.

EI-MS (M+1): 256.

### EXAMPLE 50

### Preparation of Compound 50: 1,1′-(9H-fluorene-2,7-diyl)dithiourea

Compound 50 was prepared in a manner similar to that described in Example 32.

EI-MS (M+1): 315.

### EXAMPLE 51

### Preparation of Compound 51: 1-(7-bromo-9H-fluoren-2-yl)-3-methylthiourea

Compound 51 was prepared in a manner similar to that described in Example 32.

EI-MS (M+1): 333, 335.

### EXAMPLE 52

### Preparation of Compound 52: 1-(7-bromo-9H-fluoren-2-yl)-3-ethylthiourea

Compound 52 was prepared in a manner similar to that described in Example 32.

EI-MS (M+1): 347, 349.

### EXAMPLE 53

### Preparation of Compound 53: 1-(7-bromo-9H-fluoren-2-yl)-3-propylthiourea

Compound 53 was prepared in a manner similar to that described in Example 32.

EI-MS (M+1): 361, 363.

### EXAMPLE 54

### Preparation of Compound 54: 1-(7-bromo-9H-fluoren-2-yl)-3-butylthiourea

Compound 54 was prepared in a manner similar to that described in Example 32.

EI-MS (M+1): 375, 377.

### EXAMPLE 55

### Preparation of Compound 55: 1-(7-bromo-9H-fluoren-2-yl)-3-pentyl-thiourea

Compound 55 was prepared in a manner similar to that described in Example 32.

EI-MS (M+1): 389, 391.

### EXAMPLE 56

### Preparation of Compound 56: 1-(7-bromo-9H-fluoren-2-yl)-3-hexylthiourea

Compound 56 was prepared in a manner similar to that described in Example 32.

EI-MS (M+1): 403, 405.

### EXAMPLE 57

### Preparation of Compound 57: 1-(7-bromo-9H-fluoren-2-yl)-3-heptyl-thiourea

Compound 57 was prepared in a manner similar to that described in Example 32.

EI-MS (M+1): 417, 419.

### EXAMPLE 58

### Preparation of Compound 58: 1-(7-bromo-9H-fluoren-2-yl)-3-octylthiourea

Compound 58 was prepared in a manner similar to that described in Example 32.

EI-MS (M+1): 431, 433.

### EXAMPLE 59

### Preparation of Compound 59: 1-(7-bromo-9H-fluoren-2-yl)-3-(3-methoxypropyl)thiourea

Compound 59 was prepared in a manner similar to that described in Example 32.

EI-MS (M+1): 391, 393.

### EXAMPLE 60

### Preparation of Compound 60: 1-(7-bromo-9H-fluoren-2-yl)-3-isobutyl-thiourea

Compound 60 was prepared in a manner similar to that described in Example 32.

EI-MS (M+1): 375, 377.

### EXAMPLE 61

### Preparation of Compound 61: 1-(7-bromo-9H-fluoren-2-yl)-3-(2-(dimethylamino)ethyl)thiourea

Compound 61 was prepared in a manner similar to that described in Example 32.

EI-MS (M+1): 390, 392.

### EXAMPLE 62

### Preparation of Compound 62: 1-(7-bromo-9H-fluoren-2-yl)-3-(2-(diethylamino)ethyl)thiourea

Compound 62 was prepared in a manner similar to that described in Example 32.

EI-MS (M+1): 418, 420.

### EXAMPLE 63

### Preparation of Compound 63: 1-(7-bromo-9H-fluoren-2-yl)-3-(3-(dimethylamino)propyl)thiourea

Compound 63 was prepared in a manner similar to that described in Example 32.

EI-MS (M+1): 404, 406.

### EXAMPLE 64

### Preparation of Compound 64: 1-(7-bromo-9H-fluoren-2-yl)-3-phenethyl-thiourea

Compound 64 was prepared in a manner similar to that described in Example 32.

EI-MS (M+1): 423, 425.

### EXAMPLE 65

### Preparation of Compound 65: 1-(7-bromo-9H-fluoren-2-yl)-3-(3-phenylpropyl)thiourea

Compound 65 was prepared in a manner similar to that described in Example 32.

EI-MS (M+1): 437, 439.

### EXAMPLE 66

### Preparation of Compound 66: 1-(7-bromo-9H-fluoren-2-yl)-3-(4-phenylbutyl)thiourea

Compound 66 was prepared in a manner similar to that described in Example 32.

EI-MS (M+1): 451, 453.

### EXAMPLE 67

### Preparation of Compound 67: 1-benzyl-3-(7-bromo-9H-fluoren-2-yl)thiourea

Compound 67 was prepared in a manner similar to that described in Example 32.

EI-MS (M+1): 430, 432.

### EXAMPLE 68

### Preparation of Compound 68: 1-(7-bromo-9H-fluoren-2-yl)-3-phenyl-thiourea

Compound 68 was prepared in a manner similar to that described in Example 32.

EI-MS (M+1): 394, 396.

### EXAMPLE 69

### Preparation of Compound 69: 1-(7-bromo-9H-fluoren-2-yl)-3-(pyridin-3-yl)thiourea

Compound 69 was prepared in a manner similar to that described in Example 32.

EI-MS (M+1): 395, 397.

### EXAMPLE 70

### Preparation of Compound 70: 1-(7-bromo-9H-fluoren-2-yl)-3-(4-morpholinophenyl)thiourea

Compound 70 was prepared in a manner similar to that described in Example 32.

EI-MS (M+1): 480, 482.

### EXAMPLE 71

### Preparation of Compound 71: 1-(7-bromo-9H-fluoren-2-yl)-3-(naphthalen-1-yl)thiourea

Compound 71 was prepared in a manner similar to that described in Example 32.

EI-MS (M+1): 445, 447.

### EXAMPLE 72

### Preparation of Compound 72: N-(7-thioureido-9H-fluoren-2-yl)butyramide

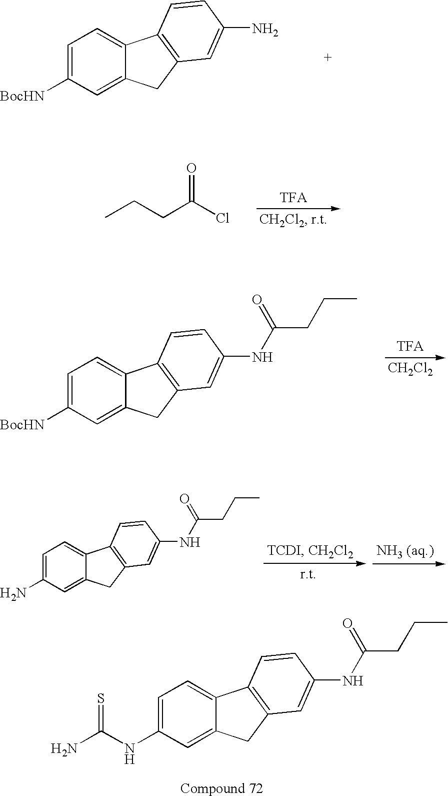

Triethylamine (37 mg, 0.37 mmol) was added to a solution of (7-amino-9H-fluoren-2-yl)-carbamic acid tert-butyl ester (100 mg, 0.34 mmol) and n-butyryl chloride (36 mg, 0.34 mmol) in dichloromethane (5 mL). The reaction mixture was stirred at room temperature for 4 hours, it was then quenched with excess saturated ammonium chloride aqueous solution (30 mL), followed by extraction with dichloromethane (30 mL×3). The organic layers were combined, washed with brine, and concentrated under vacuum. The residue was subjected to column chromatography on silica gel to give (7-butyrylamino-9H-fluoren-2-yl)-carbamic acid tert-butyl ester (99 mg, 0.27 mmol, yield: 80%) as a white solid.

Trifluoroacetic acid (TFA, 2.0 mL, 26.3 mmol) was added to a solution of (7-butyrylamino-9H-fluoren-2-yl)-carbamic acid tert-butyl ester (99 mg, 0.27 mmol) in 2 mL dichloromethane. The reaction mixture was stirred at room temperature for 1 hour. It was then quenched with water (30 mL), followed by extraction with ethyl acetate (30 ml×3). The organic layers were combined, washed with brine, and concentrated under vacuum. The residue thus obtained was subjected to column chromatography on silica gel to give N-(7-amino-9H-fluoren-2-yl)-butyramide (69 mg, 0.26 mmol, yield: 95%) as a yellow solid.

A solution of N-(7-amino-9H-fluoren-2-yl)-butyramide (69 mg, 0.26 mmol) and thiocarbonyl diimidazole (55 mg, 0.30 mmol) in dichloromethane (2 mL) was stirred at room temperature for 2 hours. After a 25% ammonia aqueous solution (2.0 mL, excess) was added, the reaction mixture was stirred at room temperature overnight. The solvent was removed and then the residue thus obtained was purified by silica gel column chromatography eluding with methanol-dichloromethane to give N-(7-thioureido-9H-fluoren-2-yl)-butyramide (compound 72) (75 mg, 0.23 mmol, yield: 90%) as a white solid.

EI-MS (M+1): 326.

### EXAMPLE 73

### Preparation of Compound 73: N-(7-thioureido-9H-fluoren-2-yl)-cyclohexanecarboxamide

Compound 73 was prepared in a manner similar to that described in Example 72.

EI-MS (M+1): 366.

### EXAMPLE 74

### Preparation of Compound 74: N-(7-thioureido-9H-fluoren-2-yl)isoxazole-5-carboxamide

Compound 74 was prepared in a manner similar to that described in Example 72.

EI-MS (M+1): 351.

### EXAMPLE 75

### Preparation of Compound 75: tert-butyl 7-thioureido-9H-fluoren-2-ylcarbamate

Compound 75 was prepared in a manner similar to that described in Example 72.

EI-MS (M+1): 356.

### EXAMPLE 76

### Preparation of Compound 76: 1-(3-(benzyloxy)phenyl)imidazolidine-2-thione

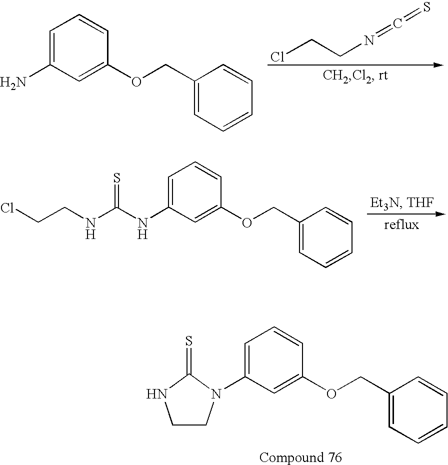

2-Chloroethyl isothiocyanate (293 mg, 2.4 mmol) was added to a solution of 3-benzyloxy-phenylamine (398 mg, 2.0 mmol) in dichloromethane (4 mL). The reaction mixture was stirred at room temperature overnight, it was quenched with water (30 mL), followed by extraction with dichloromethane (30 mL×3). The organic layers were combined, washed with brine, and concentrated under vacuum. The residue thus obtained was subjected to column chromatography on silica gel to give 1-(3-benzyloxyphenyl)-3-(2-chloro-ethyl)-thiourea (627 mg, 1.96 mmol, yield: 98%) as colorless oil.

Triethylamine (2.0 mL, excess) was added to a solution of 1-(3-benzyloxyphenyl)-3-(2-chloro-ethyl)-thiourea (187 mg, 0.58 mmol) in dry THF (3 mL). The reaction mixture was stirred at refluxing temperature for 6 hours. It was then quenched with a saturated ammonium chloride aqueous solution (30 mL), followed by extraction with ethyl acetate (30 mL×3). The organic layers were combined, washed with brine, and concentrated under vacuum. The residue thus obtained was subjected to column chromatography on silica gel to give 1-(3-benzyloxy-phenyl)-imidazolidine-2-thione (compound 76) as a white solid (150 mg, 0.52 mmol, yield: 90%).

EI-MS (M+1): 285.

### EXAMPLE 77

### Preparation of Compound 77: 1-(3-(benzyloxy)phenyl)-3-butyl-imidazolidine-2-thione

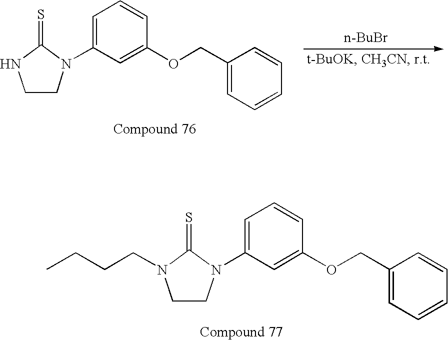

A suspension of Compound 76, i.e., 1-(3-benzyloxy-phenyl)-imidazolidine-2-thione (71 mg, 0.25 mmol) and potassium tert-butoxide (56 mg, 0.50 mmol) in acetonitrile (1 mL) was cooled in an ice bath and stirred at 0° C. for 30 minutes, followed by addition of a solution of n-butyl bromide (41 mg, 0.30 mmol) in acetonitrile (1 mL). After 5 minutes, the ice bath was removed and the reaction mixture was stirred at room temperature for 3 hours. The reaction was then quenched with water, followed by extraction with ethyl acetate (20 mL×3). The organic layers were combined and washed with brine, dried over anhydrous magnesium sulfate, and concentrated under reduced pressure. The crude mixture thus obtained was purified with silica gel column chromatography to yield 1-(3-benzyloxy-phenyl)-3-butyl-imidazolidine-2-thione (compound 77) as yellow oil (59 mg, 0.18 mmol, yield: 72%).

EI-MS (M+1): 341.

### EXAMPLE 78

### Preparation of Compound 78: 1-(3-benzyloxy-phenyl)-3-(3-phenyl-propyl)-imidazolidine-2-thione

Compound 78 was prepared in a manner similar to that described in Example 77.

EI-MS (M+1): 403.

### EXAMPLE 79

### Preparation of Compound 79: 1-[3-(5-phenyl-pentyloxy)-phenyl]-imidazolidine-2-thione

Compound 79 was prepared in a manner similar to that described in Example 76.

EI-MS (M+1): 341.

### EXAMPLE 80

### Preparation of Compound 80: 1-butyl-3-[3-(5-phenyl-pentyloxy)-phenyl]imidazolidine-2-thione

Compound 80 was prepared in a manner similar to that described in Example 77.

EI-MS (M+1): 397.

### EXAMPLE 81

### Preparation of Compound 81: 1-[3-(5-phenyl-pentyloxy)-phenyl]-3-(3-phenyl-propyl)-imidazoline-2-thione

Compound 81 was prepared in a manner similar to that, described in Example 77.

EI-MS (M+1): 459.

### EXAMPLE 82

### Preparation of Compound 82: {3-[5-(2,6-dichloro-phenoxy)pentyloxy]-phenyl}-thiourea

Compound 82 was prepared in a manner similar to that described in Example 7.

EI-MS (M+1): 400.

### EXAMPLE 83

### Preparation of Compound 83: {3-[5-(4-fluoro-phenoxy)-pentyloxy]phenyl}-thiourea

Compound 83 was prepared in a manner similar to that described in Example 7.

EI-MS (M+1): 349.

### EXAMPLE 84

### Preparation of Compound 84: {3-[5-(2-chloro-4-methoxy-phenoxy)pentyloxy]-phenyl}-thiourea

Compound 84 was prepared in a manner similar to that described in Example 7.

EI-MS (M+1): 395.

### EXAMPLE 85

### Preparation of Compound 85: {3-[5-(4-chloro-phenoxy)-pentyloxy]-phenyl}-thiourea

Compound 85 was prepared in a manner similar to that described in Example 7.

EI-MS (M+1): 365.

### EXAMPLE 86

### Preparation of Compound 86: {3-[5-(2,4-difluorophenoxy)-pentyloxy]-phenyl}-thiourea

Compound 86 was prepared in a manner similar to that described in Example 7.

EI-MS (M+1): 367.

### EXAMPLE 87

### Preparation of Compound 87: {3-[5-(2,6-dichloro-4-fluoro-phenoxy)pentyloxy]-phenyl}-thiourea

Compound 87 was prepared in a manner similar to that described in Example 7.

EI-MS (M+1): 418.

### EXAMPLE 88

### Preparation of Compound 88: {3-[5-(pyridin-4-yloxy)-pentyloxy]-phenyl}-thiourea

Compound 88 was prepared in a manner similar to that described in Example 7.

EI-MS (M+1): 332.

### EXAMPLE 89

### Preparation of Compound 89: {3-[5-(pyridin-3-yloxy)-pentyloxy]-phenyl}-thiourea

Compound 89 was prepared in a manner similar to that described in Example 7.

EI-MS (M+1): 332.

### EXAMPLE 90

### Preparation of Compound 90: {3-[5-(pyrimidin-4-yloxy)-pentyloxy]-phenyl}-thiourea

Compound 90 was prepared in a manner similar to that described in Example 7.

EI-MS (M+1): 333.

### EXAMPLE 91

### Preparation of Compound 91: 4-[5-(3-thioureido-phenoxy)-pentyloxy]-benzoic acid

Compound 91 was prepared in a manner similar to that described in Example 7.

EI-MS (M+1): 375.

### EXAMPLE 92

### Preparation of Compound 92: {3-[5-(4-dimethylamino-phenoxy)-pentyloxy]-phenyl}-thiourea

Compound 92 was prepared in a manner similar to that described in Example 7.

EI-MS (M+1): 374.

### EXAMPLE 93

### Preparation of Compound 93: {3-[5-(4-diethylamino-phenoxy)-pentyloxy]-phenyl}-thiourea

Compound 93 was prepared in a manner similar to that described in Example 7.

EI-MS (M+1): 402.

### EXAMPLE 94

### Preparation of Compound 94: {3-[5-(4-morpholin-4-yl-phenoxy)-pentyloxy]-phenyl}-thiourea

Compound 94 was prepared in a manner similar to that described in Example 7.

EI-MS (M+1): 416.

### EXAMPLE 95

### Preparation of Compound 95: {3-[5-(4-piperidin-1-yl-phenoxy)pentyloxy]-phenyl}-thiourea

Compound 95 was prepared in a manner similar to that described in Example 7.

EI-MS (M+1): 414.

### EXAMPLE 96

### Preparation of Compound 96: (3-{5-[4-(4-methyl-piperazin-1-yl)phenoxy]-pentyloxy}-phenyl)-thiourea

Compound 96 was prepared in a manner similar to that described in Example 7.

EI-MS (M+1): 429.

### EXAMPLE 97

### Preparation of Compound 97: {3-[5-(2-methoxy-phenoxy)-pentyloxy]-phenyl}-thiourea

Compound 97 was prepared in a manner similar to that described in Example 7.

EI-MS (M+1): 361.

### EXAMPLE 98

### Preparation of Compound 98: {3-[5-(3-methoxy-phenoxy)-pentyloxy]-phenyl}-thiourea

Compound 98 was prepared in a manner similar to that described in Example 7.

EI-MS (M+1): 361.

### EXAMPLE 99

### Preparation of Compound 99: {3-[5-(3,4,5-trimethoxy-phenoxy)pentyloxy]-phenyl}-thiourea

Compound 99 was prepared in a manner similar to that described in Example 7.

EI-MS (M+1): 421.

### EXAMPLE 100

### Preparation of Compound 100: {3-[5-(4-pyrrolidin-1-yl-phenoxy)-pentyloxy]-phenyl}-thiourea

Compound 100 was prepared in a manner similar to that described in Example 7.

EI-MS (M+1): 400.

### EXAMPLE 101

### Preparation of Compound 101: {3-[5-(4′-methoxy-biphenyl-4-yloxy)-pentyloxy]-phenyl}-thiourea

Compound 101 was prepared in a manner similar to that described in Example 7.

EI-MS (M+1): 437.

### EXAMPLE 102

### Preparation of Compound 102: {3-[5-(4′-methyl-biphenyl-4-yloxy)pentyloxy]-phenyl}-thiourea

Compound 102 was prepared in a manner similar to that described in Example 7.

EI-MS (M+1): 421.

### EXAMPLE 103

### Preparation of Compound 103: {3-[5-(4′-chloro-biphenyl-4-yloxy)pentyloxy]-phenyl}-thiourea

Compound 103 was prepared in a manner similar to that described in Example 7.

EI-MS (M+1): 441.

### EXAMPLE 104

### Preparation of Compound 104: {3-[5-(4′-bromo-biphenyl-4-yloxy)pentyloxy]-phenyl}-thiourea

Compound 104 was prepared in a manner similar to that described in Example 7.

EI-MS (M+1): 485, 487.

### EXAMPLE 105

### Preparation of Compound 105: {3-[5-(naphthalen-1-yloxy)-pentyloxy]-phenyl}-thiourea

Compound 105 was prepared in a manner similar to that described in Example 7.

EI-MS (M+1): 381.

### EXAMPLE 106

### Preparation of Compound 106: {3-[5-(naphthalen-2-yloxy)-pentyloxy]-phenyl}-thiourea

Compound 106 was prepared in a manner similar to that described in Example 7.

EI-MS (M+1): 381.

### EXAMPLE 107

### Preparation of Compound 107: {3-[5-(4-thiophen-3-yl-phenoxy)pentyloxy]-phenyl}-thiourea

Compound 107 was prepared in a manner similar to that described in Example 7.

EI-MS (M+1); 413.

### EXAMPLE 108

### Preparation of Compound 108: {3-[5-(4-cyano-phenoxy)-pentyloxy]phenyl}-thiourea

Compound 108 was prepared in a manner similar to that described in Example 7.

EI-MS (M+1): 356.

### EXAMPLE 109

### Preparation of Compound 109: {3-[5-(3-cyano-phenoxy)pentyloxy]-phenyl}-thiourea

Compound 109 was prepared in a manner similar to that described in Example 7.

EI-MS (M+1): 356.

### EXAMPLE 110

### Preparation of Compound 110: {3-[5-(2-cyano-phenoxy)pentyloxy]-phenyl}-thiourea

Compound 110 was prepared in a manner similar to that described in Example 7.

EI-MS (M+1): 356.

### EXAMPLE 111

### Preparation of Compound 111: {3-[5-(2,6-dichloro-4-methyl-phenoxy)pentyloxy]-phenyl}-thiourea

Compound 111 was prepared in a manner similar to that described in Example 7.

EI-MS (M+1): 414.

### EXAMPLE 112

### Preparation of Compound 112: {3-[5-(4-trifluoromethyl-phenoxy)-pentyloxy]-phenyl}-thiourea

Compound 112 was prepared in a manner similar to that described in Example 7.

EI-MS (M+1): 399.

### EXAMPLE 113

### Preparation of Compound 113: [3-(3-phenoxy-propoxy)-phenyl]-thiourea

Compound 113 was prepared in a manner similar to that described in Example 7.

EI-MS (M+1): 303.

### EXAMPLE 114

### Preparation of Compound 114; [3-(4-phenoxy-butoxy)-phenyl]-thiourea

Compound 114 was prepared in a manner similar to that described in Example 7.

EI-MS (M+1): 317.

### EXAMPLE 115

### Preparation of Compound 115: [3-(6-phenoxy-hexyloxy)-phenyl]-thiourea

Compound 115 was prepared in a manner similar to that described in Example 7.

EI-MS (M+1): 345.

### EXAMPLE 116

### Preparation of Compound 116: [3-(7-phenoxy-heptyloxy)-phenyl]-thiourea

Compound 116 was prepared in a manner similar to that described in Example 7.

EI-MS (M+1): 359.

### EXAMPLE 117

### Preparation of Compound 117: {3-[3-(biphenyl-4-yloxy)-propoxy]-phenyl}-thiourea

Compound 117 was prepared in a manner similar to that described in Example 7.

EI-MS (M+1): 379.

### EXAMPLE 118

### Preparation of Compound 118: {3-[4-(biphenyl-4-yloxy)-butoxy]-phenyl}-thiourea

Compound 118 was prepared in a manner similar to that described in Example 7.

EI-MS (M+1): 393.

### EXAMPLE 119

### Preparation of Compound 119: {3-[6-(biphenyl-4-yloxy)-hexyloxy]-phenyl}-thiourea

Compound 119 was prepared in a manner similar to that described in Example 7.

EI-MS (M+1): 421.

### EXAMPLE 120

### Preparation of Compound 120: {3-[7-(biphenyl-4-yloxy)-heptyloxy]-phenyl}-thiourea

Compound 120 was prepared in a manner similar to that described in Example 7.

EI-MS (M+1): 435.

### EXAMPLE 121

### Preparation of Compound 121: 1,1-dimethyl-3-[3-(5-phenoxy-pentyloxy)phenyl]-thiourea

Compound 121 was prepared in a manner similar to that described in Example 1.

EI-MS (M+1): 359.

### EXAMPLE 122

### Preparation of Compound 122: 1,1-Diethyl-3-[3-(5-phenoxy-pentyloxy)phenyl]-thiourea

Compound 122 was prepared in a manner similar to that described in Example 1.

EI-MS (M+1): 387.

### EXAMPLE 123

### Preparation of Compound 123: piperidine-1-carbothioic acid [3-(5-phenoxy-pentyloxy)phenyl]-amide

Compound 123 was prepared in a manner similar to that described in Example 1.

EI-MS (M+1): 399.

### EXAMPLE 124

### Preparation of Compound 124; morpholine-4-carbothioic acid [3-(5-phenoxy-pentyloxy)-phenyl]-amide

Compound 124 was prepared in a manner similar to that described in Example 1.

EI-MS (M+1): 401.

### EXAMPLE 125

### Preparation of Compound 125: 4-methyl-piperazine-1-carbothioic acid [3-(5-phenoxy-pentyloxy)phenyl]-amide

Compound 125 was prepared in a manner similar to that described in Example 1.

EI-MS (M+1): 414.

### EXAMPLE 126

### Preparation of Compound 126: {3-[5-(quinolin-6-yloxy)-pentyloxy]-phenyl}-thiourea

Compound 126 was prepared in a manner similar to that described in Example 1.

EI-MS (M+1): 382.

### EXAMPLE 127

### Preparation of Compound 127: {3-[5-(quinolin-5-yloxy)-pentyloxy]-phenyl}-thiourea

Compound 127 was prepared in a manner similar to that described in Example 1.

EI-MS (M+1): 382.

### EXAMPLE 128

### Preparation of Compound 128: {3-[5-(quinolin-4-yloxy)-pentyloxy]-phenyl}-thiourea

Compound 128 was prepared in a manner similar to that described in Example 1.

EI-MS (M+1): 382.

### EXAMPLE 129

### Preparation of Compound 129: {3-[5-(isoquinolin-5-yloxy)pentyloxy]-phenyl}-thiourea

Compound 129 was prepared in a manner similar to that described in Example 1.

EI-MS (M+1): 382.

### EXAMPLE 130

### Preparation of Compound 130: {3-[5-(quinolin-8-yloxy)-pentyloxy]-phenyl}-thiourea

Compound 130 was prepared in a manner similar to that described in Example 1.

EI-MS (M+1): 382.

### EXAMPLE 131

### Preparation of Compound 131: {3-[5-(isoquinolin-1-yloxy)-pentyloxy]-phenyl}-thiourea

Compound 131 was prepared in a manner similar to that described in Example 1.

EI-MS (M+1): 382.

### EXAMPLE 132

### Preparation of Compound 132: {3-[5-(1H-indol-4-yloxy)-pentyloxy]-phenyl}-thiourea

Compound 132 was prepared in a manner similar to that described in Example 1.

EI-MS (M+1): 370.

### EXAMPLE 133

### Preparation of Compound 133: {3-[5-(4-furan-2-yl-phenoxy)-pentyloxy]-phenyl}-thiourea

Compound 133 was prepared in a manner similar to that described in Example 1.

EI-MS (M+1): 397.

### EXAMPLE 134

### Preparation of Compound 134: {3-[5-(4-furan-3-yl-phenoxy)-pentyloxy]-phenyl}-thiourea

Compound 134 was prepared in a manner similar to that described in Example 1.

EI-MS (M+1): 397.

### EXAMPLE 135

### Preparation of Compound 135: {3-[5-(4-thiophen-2-yl-phenoxy)-pentyloxy]-phenyl}-thiourea

Compound 135 was prepared in a manner similar to that described in Example 1.

EI-MS (M+1): 413.

### EXAMPLE 136

### Preparation of Compound 136: (3-{5-[4-(5-chloro-thiophen-2-yl)phenoxy]-pentyloxy}-phenyl)-thiourea

Compound 136 was prepared in a manner similar to that described in Example 1.

EI-MS (M+1): 447.

### EXAMPLE 137

### Preparation of Compound 137: {3-[5-(4-phenoxy-phenoxy)pentyloxy]-phenyl}-thiourea

Compound 137 was prepared in a manner similar to that described in Example 1.

EI-MS (M+1): 423.

### EXAMPLE 138

### Preparation of Compound 138: {3-[5-(3-phenoxy-phenoxy)-pentyloxy]-phenyl}-thiourea

Compound 138 was prepared in a manner similar to that described in Example 1.

EI-MS (M+1): 423.

### EXAMPLE 139

### Preparation of Compound 139: {3-[5-(biphenyl-3-yloxy)-pentyloxy]-phenyl}-thiourea

Compound 139 was prepared in a manner similar to that described in Example 1.

EI-MS (M+1): 407.

### EXAMPLE 140

### Preparation of Compound 140: {3-[5-(biphenyl-2-yloxy)-pentyloxy]-phenyl}-thiourea

Compound 140 was prepared in a manner similar to that described in Example 1.

EI-MS (M+1): 407.

### EXAMPLE 141

### Preparation of Compound 141: (7-Dibenzylamino-9H-fluoren-2-yl)-thiourea

Compound 141 was prepared in a manner similar to that described in Example 39.

EI-MS (M+1): 436.

### EXAMPLE 142

### Preparation of Compound 142: (7-Benzylamino-9H-fluoren-2-yl)-thiourea

Compound 142 was prepared in a manner similar to that described in Example 39.

EI-MS (M+1): 346.

### EXAMPLE 143

### Preparation of Compound 143: {3-[5-(4-Methoxy-phenoxy)-pentyloxy]-phenyl}-thiourea

Compound 143 was prepared in a manner similar to that described in Example 7.

EI-MS (M+1): 361.

### EXAMPLE 144

### Preparation of Compound 144: {3-[5-(3,4-Dimethoxy-phenoxy)-pentyloxy]-phenyl}-thiourea

Compound 144 was prepared in a manner similar to that described in Example 7.

EI-MS (M+1): 391.

### EXAMPLE 145

### Preparation of Compound 145: {3-[5-(Pyridin-2-yloxy)pentyloxy]-phenyl}-thiourea

Compound 145 was prepared in a manner similar to that described in Example 7.

EI-MS (M+1): 382.

### EXAMPLE 146

### Preparation of Compound 146: {3-[5-(4-Pyrrol-1-yl-phenoxy)-pentyloxy]-phenyl}-thiourea

Compound 146 was prepared in a manner similar to that described in Example 7.

EI-MS (M+1): 382.

### EXAMPLE 147

### Preparation of Compound 147: {3-[5-(4-Imidazol-1-yl-phenoxy)-pentyloxy]-phenyl}-thiourea

Compound 147 was prepared in a manner similar to that described in Example 7.

EI-MS (M+1): 397.

### EXAMPLE 148

### Preparation of Compound 148: {3-[5-(4-Thiomorpholin-4-yl-phenoxy)-pentyloxy]-phenyl}-thiourea

Compound 148 was prepared in a manner similar to that described in Example 7.

EI-MS (M+1): 432.

### EXAMPLE 149

### Preparation of Compound 149: {3-[7-(Naphthalen-1-yloxy)-heptyloxy]-phenyl}-thiourea

Compound 149 was prepared in a manner similar to that described in Example 7.

EI-MS (M+1): 409.

### EXAMPLE 150

### Preparation of Compound 150: {3-[8-(Naphthalen 1-yloxy)-octyloxyl]-phenyl}-thiourea

Compound 150 was prepared in a manner similar to that described in Example 7.

EI-MS (M+1): 423.

### EXAMPLE 151

### Preparation of Compound 151: 4-[5-(3-Thioureido-phenoxy)-pentyloxy]-benzoic acid phenyl ester

Compound 151 was prepared in a manner similar to that described in Example 7.

EI-MS (M+1): 451.

### EXAMPLE 152

### Preparation of Compound 152: [4-(5-Phenyl-pentyloxy)-phenyl]-thiourea

Compound 152 was prepared in a manner similar to that described in Example 7.

EI-MS (M+1): 315.

### EXAMPLE 153

### Preparation of Compound 153: 2-[5-(3-Thioureido-phenoxy)-pentyloxy]-benzoic acid phenyl ester

Compound 153 was prepared in a manner similar to that described in Example 7.

EI-MS (M+1): 451.

### EXAMPLE 154

### Preparation of Compound 154: [2-(5-Phenyl-pentyloxy)-phenyl]-thiourea

Compound 154 was prepared in a manner similar to that described in Example 7.

EI-MS (M+1): 315.

### EXAMPLE 135

### Preparation of Compound 155: {3-[5-(3-Phenylamino-phenoxy)-pentyloxy]-phenyl}-thiourea

Compound 155 was prepared in a manner similar to that described in Example 7.

EI-MS (M+1): 422.

### EXAMPLE 156

### Preparation of Compound 156: {3-[5-(3-Benzoyl-phenoxy)-pentyloxy]-phenyl}-thiourea

Compound 156 was prepared in a manner similar to that described in Example 7.

EI-MS (M+1): 435.

### EXAMPLE 157

### Preparation of Compound 157: (3-{5-[3-(Hydroxy-phenyl-methyl)-phenoxy]-pentyloxy}-phenyl)-thiourea

Compound 157 was prepared in a manner similar to that described in Example 7.

EI-MS (M+1): 437.

### EXAMPLE 158

### Preparation of Compound 158: {3-[5-(4-Benzyl-phenoxy)-pentyloxy]-phenyl}-thiourea

Compound 158 was prepared in a manner similar to that described in Example 7.

EI-MS (M+1): 421.

### EXAMPLE 159

### Preparation of Compound 159: {3-[3-(Naphthalen-1-yloxy)-propoxy]-phenyl}-thiourea

Compound 159 was prepared in a manner similar to that described in Example 7.

EI-MS (M+1): 353.

### EXAMPLE 160

### Preparation of Compound 160: {3-[4-(Naphthalen-1-yloxy)-butoxy]-phenyl}-thiourea

Compound 160 was prepared in a manner similar to that described in Example 7.

EI-MS (M+1): 367.

### EXAMPLE 161

### Preparation of Compound 161: [4-(5-Phenoxy-pentyloxy)-phenyl]-thiourea

Compound 161 was prepared in a manner similar to that described in Example 7.

EI-MS (M+1): 381.

### EXAMPLE 162

### Preparation of Compound 162: {3-[5-(4-Methoxy-naphthalen-1-yloxy)-pentyloxy]-phenyl}-thiourea

Compound 162 was prepared in a manner similar to that described in Example 7.

EI-MS (M+1): 411.

### EXAMPLE 163

### Preparation of Compound 163: {3-[6-(Naphthalen-1-yloxy)-hexyloxy]-phenyl}-thiourea

Compound 163 was prepared in a manner similar to that described in Example 7.

EI-MS (M+1): 395.

### EXAMPLE 164

### Preparation of Compound 164: [3-(5-Naphthalen-1-yl-pentyloxy)-phenyl]-thiourea

Compound 164 was prepared in a manner similar to that described in Example 7.

EI-MS (M+1): 365.

### EXAMPLE 165

### Preparation of Compound 165: {3-[5-(4-Chloro-naphthalen-1-yloxy)-pentyloxy]-phenyl}-thiourea

Compound 165 was prepared in a manner similar to that described in Example 7.

EI-MS (M+1): 415.

### EXAMPLE 166

### Preparation of Compound 166: {3-[5-(2-Methyl-naphthalen-1-yloxy)pentyloxy]phenyl}-thiourea

Compound 166 was prepared in a manner similar to that described in Example 7.

EI-MS (M+1): 395.

### EXAMPLE 167

### Preparation of Compound 167: {3-[5-(3-Benzyl-phenoxy)-pentyloxy]-phenyl}-thiourea

Compound 167 was prepared in a manner similar to that described in Example 7.

EI-MS (M+1): 421.

### EXAMPLE 168

### Preparation of Compound 168: {3-[5-(4′-Chloro-biphenyl-2-yloxy)-pentyloxy]-phenyl}-thiourea

Compound 168 was prepared in a manner similar to that described in Example 7.

EI-MS (M+1): 441.

### EXAMPLE 169

### Preparation of Compound 169: {3-[3-(Biphenyl-2-yloxy)-propoxy]-phenyl}-thiourea

Compound 169 was prepared in a manner similar to that described in Example 7.

EI-MS (M+1): 379.

### EXAMPLE 170

### Preparation of Compound 170: {3-[4-(Biphenyl-2-yloxy)-butoxy]-phenyl}-thiourea

Compound 170 was prepared in a manner similar to that described in Example 7.

EI-MS (M+1): 393.

### EXAMPLE 171

### Preparation of Compound 171: [3-(6-Naphthalen-1-yl-hexyloxy)-phenyl]-thiourea

Compound 171 was prepared in a manner similar to that described in Example 7.

EI-MS (M+1): 379.

### EXAMPLE 172

### Preparation of Compound 172: {4-[5-(2,4-Dichloro-phenoxy)-pentyloxy]-phenyl}-thiourea

Compound 172 was prepared in a manner similar to that described in Example 7.

EI-MS (M+1): 340.

### EXAMPLE 173

### Preparation of Compound 173: {4-[5-(2,4-Difluoro-phenoxy)-pentyloxy]phenyl}-thiourea

Compound 173 was prepared in a manner similar to that described in Example 7.

EI-MS (M+1): 367.

### EXAMPLE 174

### Preparation of Compound 174: {3-[5-(4′-Fluoro-biphenyl-2-yloxy)-pentyloxy]-phenyl}-thiourea

Compound 174 was prepared in a manner similar to that described in Example 7.

EI-MS (M+1): 425.

### EXAMPLE 175

### Preparation of Compound 175: {3-[5-(4′-Trifluoromethyl-biphenyl-2-yloxy)-pentyloxy]-phenyl}-thiourea

Compound 175 was prepared in a manner similar to that described in Example 7.

EI-MS (M+1): 475.

### EXAMPLE 176

### Preparation of Compound 176: {3-[5-(4′-Methoxy-biphenyl-2-yloxy)-pentyloxy]-phenyl}-thiourea

Compound 176 was prepared in a manner similar to that described in Example 7.

EI-MS (M+1): 437.

### EXAMPLE 177

### Preparation of Compound 177: {3-[5-(4′-Methyl-biphenyl-2-yloxy)-pentyloxy]-phenyl}-thiourea

Compound 177 was prepared in a manner similar to that described in Example 7.

EI-MS (M+1): 421.

### EXAMPLE 178

### Preparation of Compound 178: {3-[5-(3′-Methyl-biphenyl-2-yloxy)-pentyloxy]-phenyl}-thiourea

Compound 178 was prepared in a manner similar to that described in Example 7.

EI-MS (M+1): 421.

### EXAMPLE 179

### Preparation of Compound 179: {3-[5-(3,5-Difluoro-biphenyl-2-yloxy)-pentyloxy]-phenyl}-thiourea

Compound 179 was prepared in a manner similar to that described in Example 7.

EI-MS (M+1): 443.

### EXAMPLE 180

### Preparation of Compound 180: {3-[5-(Naphthalen-1-ylamino)-pentyloxy]-phenyl}-thiourea

Compound 180 was prepared in a manner similar to that described in Example 7.

EI-MS (M+1): 380.

### EXAMPLE 181

### Preparation of Compound 181: {3-[5-(2-Cyclohexyl-phenoxy)-pentyloxy]-phenyl}-thiourea

Compound 181 was prepared in a manner similar to that described in Example 7.

EI-MS (M+1): 413.

### EXAMPLE 182

### Preparation of Compound 182: {3-[5-(4-Cyclohexyl-phenoxy)-pentyloxy]-phenyl}-thiourea

Compound 182 was prepared in a manner similar to that described in Example 7.

EI-MS (M+1): 413.

### EXAMPLE 183

### Preparation of Compound 183: {3-[5-(2-Furan-2-yl-phenoxy)-pentyloxy]-phenyl}-thiourea

Compound 183 was prepared in a manner similar to that described in Example 7.

EI-MS (M+1): 397.

### EXAMPLE 184

### Assay for Inhibition of HCV Replication

Dulbecco's modified Eagle's medium (DMEM) high glucose, fetal bovine serum (FBS), G418 (geneticin), and blasticidin were purchased from Invitrogen (Carlsbad, Calif.). A reporter cell line, Ava5-EG(Δ4AB)SEAP, for HCV drug screening was derived from HCV replicon cells (Ava5). See, e.g., Lee et al., Anal. Biochem. 316:162-70 and Lee et al., J. Viral. Methods 116:27-33. EG(Δ4AB)SEAP is a reporter gene consisting of enhanced green fluorescent protein (EG), an NS3-NS4A protease decapeptide recognition sequence (Δ4AB), and secreted alkaline phosphatase (SEAP). See, e.g., Lee et al., Anal. Biochem. 316:162-70. A reporter gene, EG(Δ4AB)SEAP, was stably integrated in the Ava5 cells to generate Ava5-EG(Δ4AB)SEAP cells. The cells were cultured in a medium containing 500 μg/ml G418 (geneticin) and 10 μg/ml blasticidin in a 5% CO₂ incubator.

Ava5-EG(Δ4AB)SEAP cells were seeded in 96-well plates (5×10³ cells/100 μl/well). After incubation for 1 day, the cells were treated with various concentrations of a test compound for 48 hours. Each culture medium was replenished with a fresh medium containing the test compound at the same concentration to remove the accumulated SEAP. The cells were then incubated for another 24 hours. The culture medium was collected and subjected to SEAP activity assays. The SEAP activities were measured using the Phospha-Light assay kit (Tropix, Foster, Calif., USA) according to manufacturer's instructions. Of note, SEAP activity in the culture medium, can be used to reflect anti-HCV activity. See, e.g., Lee et al., J. Virol. Methods 116:27-33.

Compounds 1-42, 45-62, 64-91, 93-135, and 137-183 were tested for their efficacy in inhibiting HCV replication. Unexpectedly, 119 test compounds showed low EC₅₀ values (i.e., the concentration of a test compound at which 50% HCV replication is inhibited) between 0.001 μM and 1 μM. Among them, 63 test compounds showed EC₅₀ values as low as between 0.001 μM and 0.1 μM.

### EXAMPLE 185

### Cytotoxicity Assay

Cell viability was determined by the MTS assay similar to that described in Cory et al., Cancer Commun. 3:207-12. In short Ava5-EG(Δ4AB)SEAP cells were seeded in 96-well plates (5×10³ cells/100 μl/well). 100 μL/well solution containing phenol red-free DMEM, MTS (tetrazolium compound [3-(4,5-dimethylthiozol-2-yl)>5-(3-carboxymethoxyphenyl)-2-(4-sulfophenyl)-2H-tetrazolium, inner salt]; Promega, Madison, Wis.) and phenazine methosulfate (PMS; Sigma, St. Louis, Mo.) at a ratio of 80:20:1 to each well. The cells were incubated with test compounds for 1-4 hours at 37° C. in a humidified, 5% CO₂ incubator and the absorbance was then measured at 490 nm.

Compounds 1-42, 45-62, 64-91, 93-135, and 137-183 were tested in the above cytotoxicity assay. Unexpectedly, all test compounds showed CC₅₀ values (i.e., the concentration of a test compound at which 50% of the cells are killed) above 1 μM. Specifically, 67 of the tested compounds showed CC₅₀ values above 50 μM, 88 of the tested compounds showed CC₅₀ values between 10 μM and 50 μM, and 23 of the test compounds showed CC₅₀ values between 1 μM and 10 μM. Most of the effective compounds exerted little cytotoxicity.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 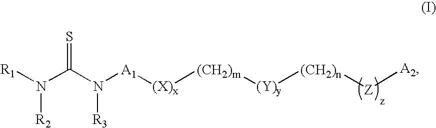
wherein
each of R₁, R₂, and R₃, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₂-C₂₀ heterocycloalkenyl, aryl, or heteroaryl; or R₁ and R₂, together with the nitrogen atom to which they are bonded, are C₃-C₂₀ heterocycloalkyl; or R₂ and R₃, together with the two nitrogen atoms to which they are bonded and the carbon atom bonded to both of the two nitrogen atoms, are C₃-C₂₀ heterocycloalkyl;
each of A₁ and A₂, independently, is aryl or heteroaryl;
each of X, Y, and Z, independently, is O, S, S(O), S(O)₂, N(Rₐ), C(RₐR_{b}), C₁-C₁₀ alkyl C₂-C₁₀ to alkenyl, C₂-C₁₀alkynyl, C₃-C₂₀ cycloalkyl, C₁-C₂₀ heterocycloalkyl, aryl, or heteroaryl, in which each of Rₐ and R_{b}, independently, is H, C₁-C₁₀ alkyl, C₃-C₂₀cycloalkyl, C₁-C₂₀ heterocycloalkyl, aryl, or heteroaryl;
each of m and n, independently, is 1, 2, 3, 4, or 5; and
each of x, y, and z, independently, is 0 or 1.

2. The compound of claim 1, wherein x is 1, y is 0, and z is 0.

3. The compound of claim 2, wherein X is O or NH.

4. The compound of claim 3, wherein a is phenylene and A₂ is phenyl.

5. The compound of claim 4, wherein each of R₁, R₂, and R₃, independently, is H or C₁-C₁₀ alkyl optionally substituted with aryl.

6. The compound of claim 1, wherein the compound is one of compounds 1-6, 15, 17, 18, 21, 22, 23, 152, 154, 164, and 171.

7. The compound of claim 1, wherein x is 1, y is 0, and z is 1.

8. The compound of claim 7, wherein X is O and Z is O.

9. The compound of claim 8, wherein A₁ is phenylene and A₂ is aryl or heteroaryl, optionally substituted with halo, aryl, heteroaryl, CN, OR, COOR, or NRR′, in which each of R and R′ independently, is H, C₁-C₁₀ alkyl, or aryl.

10. The compound of claim 9, wherein each of R₁, R₂, and R₃ is H, or R₁ and R₂, together with the nitrogen atom to which they are bonded, are C₃-C₂₀heterocycloalkyl.

11. The compound of claim 8, wherein A₁ is phenylene and A₂ is phenyl, naphthyl, or pyridinyl, optionally substituted with halo, alkyl, cycloalkyl, heterocycloalkyl, aryl, heteroaryl CN, OR, COR, COOR, or NRR′, in which each of R and R′ independently, is H, C₁-C₁₀ alkyl, or aryl.

12. The compound of claim 11, wherein each of R₁, R₂, and R₃ is H, or R₁ and R₂, together with the nitrogen atom to which they are bonded, are C₃-C₂₀heterocycloalkyl.

13. The compound of claim 1, wherein the compound is one of compounds 7-9, 12, 82-87, 93-120, 126-129, 132-135, 137-140, 143-146, 148-151, 153-161, 163, 165-170, and 172-183.

14. The compound of claim 1, wherein x is 1, y is 1, and z is 1.

15. The compound of claim 14, wherein X is O, Y is C(RₐR_{b}), and Z is O, in which each of Rₐ, and R_{b}, independently, is C₁-C₁₀ alkyl.

16. The compound of claim 15, wherein A₁ is phenylene and A₂ is phenyl optionally substituted with aryl.

17. The compound of claim 16, wherein each of R₁, R₂, and R₃ is H.

18. The compound of claim 1, wherein the compound is one of compounds 10, 11, 13, and 14.

19. The compound of claim 1, wherein A₁ is phenylene and A₂ is aryl or heteroaryl, optionally substituted with halo, alkyl, cycloalkyl, heterocycloalkyl, aryl, heteroaryl, CN, OR, COR, COOR, or NRR′, in which each of R and R′ independently, is H, C₁-C₁₀ alkyl, or aryl.

20. The compound of claim 19, wherein A₂ is phenyl, naphthyl, or pyridinyl, optionally substituted with halo, alkyl, cycloalkyl, heterocycloalkyl, aryl, heteroaryl, CN, OR, COR, COOR, or NRR′, in which each of R and R′ independently, is H, C₁-C₁₀ alkyl, or aryl.

21. The compound of claim 20, wherein each of R₁, R₂, and R₃ is H, or R₁ and R₂, together with the nitrogen atom to which they are bonded, are C₃-C₂₀heterocycloalkyl.

22. The compound of claim 1, wherein each of R₁, R₂, and R₃ is H.

23. A compound of formula (I): 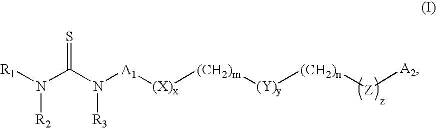
wherein
R₁ is H, C₁-C₁₀ alkyl, C₂-C₁₀ alkenyl, C₃-C₁₀ alkynyl, C₃-C₂₀ cycloalkyl, C₃-C₂₀cycloalkenyl, C₁-C₁₀ heterocycloalkyl, C₁-C₁₀ heterocycloalkenyl, aryl, or heteroaryl;
each of R₂ and R₃, independently, is C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; or R₂ and R₃, together with the two nitrogen atoms to which they are bonded and the carbon atom bonded to both of the two nitrogen atoms, are C₃-C₂₀ heterocycloalkyl;
each of A₁ and A₂, independently, is aryl or heteroaryl;
each of X, Y, and Z, independently, is O, S, S(O), S(O)₂, N(Rₐ), C(RₐR_{b}), C₁-C₁₀ alkyl, C₂-C₁₀ alkenyl, C₂-C₁₀ alkynyl, C₃-C₂₀ cycloalkyl, C₁-C₂₀ heterocycloalkyl, aryl, or heteroaryl, in which each of Rₐ and R_{b}, independently, is H, C₁-C₁₀ alkyl, C₃-C₂₀cycloalkyl, C₁-C₂₀heterocycloalkyl, aryl, or heteroaryl;
each of m and n, independently, is 0, 1, 2, 3, 4, or 5; and
each of x, y, and z, independently, is 0 or 1.

24. The compound of claim 23, wherein x is 1, y is 0, and z is 0.

25. The compound of claim 24, wherein X is O.

26. The compound of claim 25, wherein and R₂ and R₃, together with the two nitrogen atoms to which they are bonded and the carbon atom bonded to both of the two nitrogen atoms, are C₃-C₂₀ heterocycloalkyl;

27. The compound of claim 26, wherein A₁ is phenylene and A₂ is phenyl.

28. The compound of claim 27, wherein R₁ is H or C₁-C₁₀ alkyl optionally substituted with aryl.

29. A method for treating hepatitis C virus infection, comprising administering to a subject in need thereof an effective amount of a compound of formula (I): 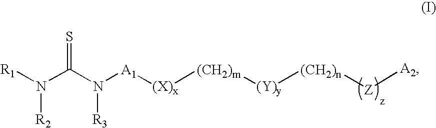
wherein
each of R₁, R₂, and R₃, independently, is H C₁-C₁₀alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; or R₁ and R₂, together with the nitrogen atom to which they are bonded, are heterocycloalkyl; or R₁ and R₃, together with the two nitrogen atoms to which they are bonded and the carbon atom bonded to both, of the two nitrogen atoms, are C₃-C₂₀ heterocycloalkyl;
each of A₁ and A₂, independently, is aryl or heteroaryl;
each of X, Y, and Z, independently, is O, S, S(O), S(O)₂, N(Rₐ), C(RₐR_{b}), C₁-C₁₀ alkyl, C₂-C₁₀ alkenyl, C₂-C₁₀ alkynyl, C₃-C₂₀ cycloalkyl, C₁-C₂₀ heterocycloalkyl, aryl, or heteroaryl, in which each of Rₐ and R_{b}, independently, is H, C₁-C₁₀ alkyl, C₃-C₂₀cycloalkyl, C₁-C₂₀ heterocycloalkyl, aryl, or heteroaryl;
each of m and n, independently, is 1, 2, 3, 4, or 5; and
each of x, y, and z, independently, is 0 or 1.

30. The method of claim 29, wherein x is 1, y is 0, and z is 0.

31. The method of claim 30, wherein X is O or NH.

32. The method of claim 31, wherein A₁ is phenylene and A₂ is phenyl.

33. The method of claim 29, wherein A₁ is phenylene and A₂ is phenyl optionally substituted with aryl.

34. A method for treating hepatitis C virus infection, comprising administering to a subject in need thereof an effective amount of a compound of formula (I): 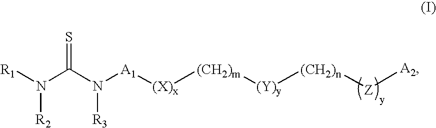
wherein
R₁ is H, C₁-C₁₀ alkyl C₂-C₁₀ alkenyl, C₂-C₁₀ alkynyl C₃-C₂₀cycloalkyl, C₃-C₂₀cycloalkenyl, C₁-C₂₀ heterocycloalkyl C₁-C₂₀ heterocycloalkenyl, aryl, or heteroaryl;
each of R₂ and R₃, independently, is C₁-C₁₀ alkyl, C₁-C₁₀ alkenyl C₂-C₁₀ alkynyl, C₃-C₂₀ cycloalkyl, C₃-C₂₀ cycloalkenyl, C₁-C₂₀ heterocycloalkyl C₁-C₂₀heterocycloalkenyl aryl, or heteroaryl; or R₂ and R₃, together with the two nitrogen atoms to which they are bonded and the carbon atom bonded to both of the two nitrogen atoms, are C₃-C₂₀ heterocycloalkyl;
each of A₁ and A₂, independently, is aryl or heteroaryl;
each of X, Y, and Z, independently, is O, S, S(O), S(O)₂, N(Rₐ), C(RₐR_{b}), C₁-C₁₀alkyl, C₂-C₁₀ alkenyl, C₂-C₁₀ alkynyl, C₃-C₂₀ cycloalkyl, C₁-C₂₀heterocycloalkyl, aryl, or heteroaryl, in which each of Rₐ and R_{b}, independently, is H, C₁-C₁₀ alkyl, C₃-C₂₀cycloalkyl, C₁-20 heterocycloalkyl, aryl, or heteroaryl;
each of m and n, independently, is 0, 1, 2, 3, 4, or 5; and
each of x, y, and z, independently, is 0 or 1.

35. The method of claim 34, wherein, x is 1, y is 0, and z is 0.

36. The method of claim 35, wherein X is O.

37. The method of claim 36, wherein R₂ and R₃, together with the two nitrogen atoms to which they are bonded and the carbon atom bonded to both of the two nitrogen atoms, are C₃-C₂₀ heterocycloalkyl;

38. The method of claim 37, wherein A₁ is phenylene and A₂ is phenyl.
